SEQUENCE LISTING (2 sequences)

SEQ ID NO: 1 - DNA, 2076 bp, Artificial Sequence
  synthetic
atgattaaactgtctggggaaggattactgcacacgtgctatagtacaaatgatcaaatcgcccttaaaaacctgcagtctgatgttacagagaggaagttggacttctccaaggagacactggcatcacagaacacaagaatgatatcacctgtggtcatttcccggctgattgatgaaaagaaatctatggaaaatggagcaatattgccacaagccattgctcagcctcagctgtgtcccacaaagccagctttggccaggcgcgatggagtgagcatgcacaggcgatttgctttatctccagacagactgggaattttgactccatcagatgatcaaggactggagacagaaccgctgtctacaggggacaatctaggcaaaggttcccacagcggtttctcgtctatcaccattactgcccgacgtgtgggccctccagccagcagcttggtatgggacacttttagggacccactgtgtcccaagtgcaaagccaaggatgctctgttccaagagcccccagttctggctggtgatgcccatctctgtcagcacaatagacccttcacctgtacagagtcccccagcaatggctccgtagaggggatgaaggttttccaggctcactcaaggctgagtgcaaggcaggactactgggtcacccacacgaatgacaatgaggacagtttttcttcagacaattcaccatcgagaaaagtcccactggtgttcagttcttgtgtccacttccgggtgtctcagcagtgtcccaatgccatttactatttggacaagtctctttctgttccccttgagcgacctcaaattgctagccccaaaatgcacaggtctgtcctgtctctcagcctccgctgtagttcccaccagttaacagcagatggagtagacagctcagctaatggagagccaatcagcacagctctatcacaggaactctcggagggaaagcaggacctcctaggtccacaatggggccagccacaaggtggtcactggaaggagagcccagcattggtgccagtacatttggggagtggcacatgccctcggactggctcccctccactggaaaatgtcaaatttgcagacgtgggaaggaaccaggtcccagtacgaaaggagaaagaggaccatgcaacatgtaccagtagcagccacaccaaccaactctccatccatattcccggctggagttacagggctgaaacaaaagtactttctggaagcaagaagcagcagcaagaagcacaaagaactcttcctgctttcccagtgggacagaagaccattaaacacttccctcctgaaggggacagttcaccaagcagtgatggtcaacctagcatcctttcagagtccaatgagaggcaacacccatacttcatgatacccagagtccctctgcctgggttttattgccctttacaaactgggtgtgcctctctgcaggaagatggtgctgttcagatagaaacacacttccccaaagattacacgtgctgtgacttggttgtaaaactaaaggaatgtgagaagaacgaagaccccactgtcacccctgagccctcaccagcaaccccctctccatcaaccccagagggagcacagagctctgacccatcagaagatagttacgagcctctgctggcaagttctatgactttgcaggaagcactggaagtccatagacctcagttcatttctcgctcacaagaacggctgcaaaagcttaagcgcatggtacagcaaaggaaaacccaacaaaaggagagcctggggcagaagcagagcctacttcctgtccgcgccaacaagaagcagttcaccattccccatcctcttagtgataacttgttcaaacccaaagaaagatgcatttcagagaaggagatgcatatgagatctaaaagaatctataataatttgccggaagttaaaaagaaaaaagaagagcagaagaaaagaatgatcttacagagtaaccggctccgagcagaagtcttcaaaaagcaattactggaccagctccttcaaaggaatgcagtttga

SEQ ID NO: 2 - DNA, 19 bp, Artificial Sequence
  synthetic
gcaacatgtaccagtagca

(19) United States
(12) Patent Application Publication
(10) Pub. No.: US 2019/0055560 A1
(43) Pub. Date: Feb. 21, 2019

(54) FATS AS A TARGET FOR TREATING TUMORS AND USES THEREOF

(71) Applicant: TIANJIN MEDICAL UNIVERSITY, Tianjin (CN)

(72) Inventors: Rongxin ZHANG, Tianjin (CN); Kai ZHANG, Tianjin (CN); Lijuan ZHANG, Tianjin (CN); Zhenyi XUE, Tianjin (CN); Yan LI, Tianjin (CN)

(21) Appl. No.: 16/134,983

(22) Filed: Sep. 19, 2018

Related U.S. Application Data
(63) Continuation-in-part of application No. PCT/CN2017/082989, filed on May 4, 2017.

(30) Foreign Application Priority Data
May 12, 2016 (CN) 201610315637.9

(51) Int. Cl. C12N 15/113; A61P 35/00

(52) U.S. Cl. CPC C12N 15/113; C12N 2310/14; A61P 35/00

(57) ABSTRACT

Disclosed are a use of FATS gene, by knocking out or inhibiting the FATS gene, in promoting macrophage polarization into M1 type and/or inhibiting macrophage polarization into M2 type, or activating and proliferating killer T cells; and a use of FATS gene or an expression product thereof in any one of i) developing and screening a functional product for tumors and ii) preparing a functional product for treatment or prevention of tumors. The present application demonstrates that FATS gene or an expression product thereof is closely related to tumors, and thus can be used as a drug target to develop tumor-related drugs. Cellular and molecular mechanisms of the FATS gene or its expression product in tumors are further demonstrated, providing an effective targeting means or a major basis for the development of tumor-related drugs.

### CROSS-REFERENCE TO RELATED APPLICATIONS

This application is a continuation of International Application No. PCT/CN2017/082989, filed on May 4, 2017 which claims the benefit of priority from Chinese Application No. 201610315637.9, filed on May 12, 2016. The content of the aforementioned applications, including any intervening amendments thereto, are incorporated herein by reference.

### TECHNICAL FIELD

The present application relates to biotechnology and in particular to FATS as a target for treating tumors and uses thereof.

### BACKGROUND OF THE PRESENT INVENTION

As a malignant tumor originating from melanocytes, melanoma is the most aggressive tumor among all skin tumors. Cells that produce pigments may be derived from various tissues including skin, mucous membrane and conjunctiva in the body. Despite of decades of continuous development of many chemotherapy drugs and methods for treating malignant tumors, the survival rate of patients with metastatic melanoma has still not increased. There are about 73,870 cases suffering from melanoma in the U.S. in 2015. Although melanoma is not as common as other skin cancers, for example, basal cell carcinoma and squamous cell carcinoma, melanoma results in a high percentage of deaths in all skin cancers. According to the extent of tumor progression, there are significant differences in terms of the survival rate of melanoma patients. Patients in early stages only need surgical resection; however, the patients in metastatic stages have only 16.6% of 5-year survival rate.

Fortunately, the understanding of melanoma in recent years provides some useful information for clinical research. The development of powerful molecular diagnostic tools and approaches lead to the discovery of various mutation, amplification or deletion of genes during the growth and survival of tumors, as well as the sensitivity response to small molecule inhibitors. This enables the development of efficient signal-transduction target and immunotherapeutic target. The unfavorable prognosis has changed after the emergence of systemic therapy. Six drugs (Ipilimumab, Vemurafenib, Dabrafenib, Trametinib, etc.) have been approved by the U.S. Food and Drug Administration (FDA) since 2011. These drugs can be used to treat melanoma via 4 different mechanisms (inhibiting CTL-associated antibody inhibitors, BRAF, MEK and PD-1 receptor).

It is found that the carcinogenic BRAF mutations promote tumor growth in up to 50% of melanomas. Mutations in BRAF and other genes (e.g., KIT) provides various approaches to systemic therapy. Targeted treatments, including the use of BRAF and MEK inhibitors, have increased the overall survival rate of melanoma patients with BRAF V600 mutation. In the past 5 to 10 years, the treatment of melanoma is of great change due to the discovery of a new type of immunoregulatory factor. The inhibition of immunoregulatory checkpoints has greatly altered the situation of melanoma treatment. Inactivation of immunoregulatory checkpoints limits the immune response of T cells in melanoma, which is a target for immunotherapy of cancers. It is very important to succeed in treatments using Ipilimumab as immunoregulatory checkpoint inhibitor and anti-PD-1 antibodies and targeting CTLA-4 and PD-1/PD-L1.

Pancreatic cancer is one of the most common malignant tumors of the digestive system. Patients usually have advanced due to an extremely low diagnosis rate of early pancreatic cancer, and the prognosis remains undesirable. In recent years, the incidence of pancreatic cancer has increased significantly. Global statistics shows that the deaths caused by pancreatic cancer take the fifth place among all cancer-related deaths. Furthermore, conventional radiotherapy and chemotherapy barely works for pancreatic cancer. Therefore, there is a need to develop new therapies such as immunotherapy to improve the effect for clinical therapy of pancreatic cancer.

A large number of studies have shown that immunotherapy of tumors is an option for patients having advanced cancer. Currently, immunotherapy is capable of destroying tumor cells, and the functional level of immune system is closely related to the prognosis and clinical therapeutic effect of tumors. It has been found that targeting tumor microenvironment (TME) has become an important strategy for anti-tumor therapies. It is known that immune cells in TME can affect the occurrence, development and invasion of tumors, as well as the final therapeutic effect.

Chromosomal fragile site is an unstable site-specific region in normal genome, including 88 common fragile sites (CFSs) and 39 rare fragile sites. CFSs are normal structures as the components of chromosome. In the case of replication abnormalities in the metaphase of cell division, the chromosomal fragile site fragile sites in the chromosome are most prone to cracks or breakpoints. CFSs are highly conserved during evolution. C10orf90 is a general fragile site established recently. It is found in initial studies that tumor suppression occurs upon over-expression of C10orf90 in several tumor cell lines and thus C10orf90 is named Fragile site-associated tumor suppressor (FATS). It has been reported that CFSs are also associated with immunity. Nevertheless, there is no report on the role of FATS gene in tumor immunity, considering that researches on FATS gene are very limited.

### SUMMARY OF THE PRESENT INVENTION

The applicant has studied the relationship between FATS gene and tumors and found that FATS gene is an important immunoregulatory factor, which plays a vital role in autoimmune diseases and tumor immunity.

In an aspect, the present application provides a use of FATS gene, by knocking out or inhibiting the FATS gene, in promoting macrophage polarization into M1 type and/or inhibiting macrophage polarization into M2 type, or activating and proliferating killer T cells.

In another aspect, the present application provides a use of FATS gene or an expression product thereof in any one of:

i) developing and screening a functional product for tumors, and

ii) preparing a functional product for treatment or prevention of tumors.

The present application further provides a functional product for treating or preventing tumors by acting on FATS gene or an expression product thereof.

The present application further provides a method of:

i) developing and screening a functional product for tumors; or

ii) preparing a functional product for treatment or prevention of tumors.

Preferably, the functional product is a product or a latent substance at least for treating, alleviating, inhibiting or regulating occurrence and progression of tumors. Further, the functional product includes a pharmaceutical such as medicine, medicament and an inhibitor. The functional product may be a single formulation and/or a composition containing an effective amount of components, wherein the composition may include a pharmaceutically acceptable carrier.

Preferably, the functional product includes the functions of down-regulating expression, transcription of the FATS gene or expression product of the FATS gene. The method comprises the steps of down-regulating expression, transcription of the FATS gene or expression product of the FATS gene. As known to one skilled in the art, the method for down-regulating expression, transcription of the FATS gene or expression product of the FATS gene includes but is not limited to one or more of:

i) at a DNA level: reducing the copy number of the FATS gene, and/or transfecting with a low-expression vector for FATS gene;

ii) at a transcriptional level: blocking or inhibiting the FATS gene expression, blocking or inactivating a promoter that regulates the FATS gene expression, activating a transcription factor that negatively regulates the FATS gene expression, and/or interfering with the FATS gene expression using a RNA interference technique;

iii) at a post-transcription level: activating the transcription of a microRNA that promotes the degradation of the FATS gene mRNA, and/or introducing a microRNA that inhibits the FATS gene expression; and

(iv) at a post-translational level: introducing a molecule that inhibits the FATS gene encoding protein; promoting the expression of a protein that negatively regulates the FATS gene expression, and/or expressing a factor and/or a protein that inhibits the FATS gene expression.

In some preferred embodiments, the functional product increases infiltration of inflammatory cell in a tumor tissue.

In another preferred embodiment, the functional product promotes an anti-tumor immunity and/or inhibits a tumor-promoting immune response in a peripheral immune organ or a tumor immune microenvironment.

In another preferred embodiment, the functional product promotes polarization of macrophages into M1 type and/or inhibits polarization of macrophages into M2 type during differentiation of macrophages.

In another preferred embodiments, the functional product is used for one or more of:

i) increasing a frequency of at least one of cytotoxic T lymphocytes, NK cells, γδ T cells, and M1 type macrophages;

ii) reducing a frequency of regulatory T lymphocytes and/or M2 type macrophages;

iii) increasing an expression of cytokine IL-12 secreted by M1 type macrophages and/or an expression of T cell proliferation-associated cytokine IL-2;

iv) increasing a killing ability of macrophages;

v) increasing a proliferative ability of T cells;

vi) reducing an expression of VEGF by macrophages;

vii) inhibiting angiogenesis in a tumor tissue;

viii) promoting polarization of macrophages into M1 type and/or inhibiting polarization of macrophages into M2 type during differentiation of bone marrow cells into macrophages.

ix) promoting an apoptosis of M2 type macrophages; and

x) activating a NF-κB signaling pathway.

In some preferred embodiments, the functional product is selected from or includes one or more of a nucleic acid inhibitor, a protein inhibitor, an antibody, a ligand, a proteolytic enzyme, a protein-binding molecule, a FATS gene deficient or silenced immune-associated cell or a differentiated cell or a construct thereof, capable of down-regulating expression or expression product of the FATS gene at a genetic or protein level.

In another preferred embodiment, the functional product is selected from or includes one or more of a small interfering RNA, a dsRNA, a shRNA, a microRNA or an antisense nucleic acid targeting the FATS gene or a transcript of the FATS gene and capable of inhibiting expression or transcription of the FATS gene; and a construct capable of expressing or forming the small interfering RNA, the dsRNA, the shRNA, the microRNA or the antisense nucleic acid.

In another preferred embodiment, the functional product is selected from includes any one of:

i) a small interfering RNA, a dsRNA, an shRNA, a microRNA or an antisense nucleic acid targeting SEQ ID NO:1 or a transcript of SEQ ID NO:1 and capable of inhibiting expression or transcription of the FATS gene;

ii) a construct capable of expressing or forming the small interfering RNA, the dsRNA, the shRNA, the microRNA or the antisense nucleic acid in i);

iii) a construct, comprising SEQ ID NO: 1 or a complementary sequence of SEQ ID NO: 1, and capable of forming an interfering molecule inhibiting expression or transcription of the FATS gene in vivo;

iv) an immune-associated cell in which SEQ ID NO: 1 is inhibited or knocked out, or a differentiated cell or a construct of the immune-associated cell;

v) a small interfering RNA, a dsRNA, an shRNA, a microRNA or an antisense nucleic acid targeting a homologous sequence or a transcript of SEQ ID NO: 1 according to codon preference in organism of a construct, and capable of inhibiting expression or transcription of the FATS gene;

vi) a construct capable of expressing or forming the small interfering RNA, the dsRNA, the shRNA, the microRNA or the antisense nucleic acid in v);

vii) a construct comprising a homologous sequence or a complementary sequence of SEQ ID NO: 1 according to codon preference of organism of the construct, and capable of forming an interfering molecule inhibiting expression or transcription of the FATS gene in vivo; and

viii) an immune-associated cell in which a homologous gene sequence of SEQ ID NO: 1 according to codon preference of organism of a construct is inhibited or knocked out, or a differentiated cell or a construct of the immune-associated cell.

In another preferred embodiment, the expression product of the FATS gene includes a protein encoded by the FATS gene.

In another preferred embodiment, the tumors include melanoma or pancreatic cancer.

The construct may be a cell (e.g., a transfected cell) or an expression vector. The homology of the homologous sequence is greater than 70%.

The FATS gene or expression product of the FATS gene is interpreted to include:

i) an original sequence or a fragment of FATS gene or expression product thereof;

ii) a conservative variant, a biologically active fragment or a derivative of FATS gene or expression product thereof;

iii) an original sequence or a fragment of FATS gene or expression product thereof according to the codon preference of organism of the construct; and

iv) a conservative variant, a biologically active fragment or a derivative of FATS gene or expression product thereof according to the codon preference of organism of the construct.

The present invention has the following advantages:

(1) it is revealed that FATS gene or its expression product is closely related to tumors, so that FATS gene or its expression product can serve as a drug target for the development of anti-tumor drugs;

(2) it is revealed that the cellular and molecular mechanisms of how the FATS gene or its expression product is effective in tumors, thus providing an effective means or important basis for the development of anti-tumor drugs.

### BRIEF DESCRIPTION OF THE DRAWINGS

FIGS. 1A-1E show inhibition of FATS gene defect on development of tumor-graft melanoma in subcutaneous B16 cells in mice. 2×10⁵ B16 cells were injected subcutaneously to the right back of mice (wild type (WT) mice, n=16; knockout (KO) mice, n=15), and tumor size was observed and measured till the 20th day. The mice were sacrificed to measure the volume and weight of the melanoma. FIG. 1A shows a growth curve of the melanoma in WT and KO mice. FIG. 1B is an image showing representative results of the melanoma in WT and KO mice. FIG. 1C shows final weights of the melanoma in WT and KO mice. FIG. 1D shows non-tumorigenicity in WT and KO mice (P=0.0083). FIG. 1E shows representative results for WT and KO mice (*, P<0.05; **, P<0.01; ***, P<0.001).

FIG. 2 shows that FATS gene defect increases infiltration of inflammatory cells into the melanoma in mice. B16 cells were injected subcutaneously into the WT and KO mice for a tumor-graft experiment, and the mice were sacrificed after 20 days. Partial tumor tissues of melanoma from the WT and KO mice were embedded with paraffin, sectioned (with thickness of 5 μm) and stained by H&E. The right two columns (“Within tumor” and “Tumor edge”) show enlarged views of the tissue image in the first columns (left) to illustrate the infiltrating inflammatory cells.

FIGS. 3A-3D show that FATS gene defect increases the frequency of T cells and γδ T cells in the spleen of mice with melanoma. B16 cells were injected subcutaneously into the WT and KO mice for a tumor-graft experiment and the mice were sacrificed after 20 days. Mononuclear cells were separated from the spleens of the WT and KO mice and analyzed using flow cytometry for detecting changes in immune cell subsets (total T cells and γδ T cells). FIG. 3A is a typical flow cytometry plot showing the frequency of total T cells in the spleens from the WT and KO mice with melanoma. FIG. 3B is a statistical chart showing the frequency of total T cells in the spleens from the WT and KO mice with melanoma. FIG. 3C is a typical flow cytometry plot showing the frequency of γδ T cells in the spleens from the WT and KO mice with melanoma. FIG. 3D is a statistical chart showing the frequency of total γδ T cells in the spleens from the WT and KO mice with melanoma (*, P<0.05; **, P<0.01; ***, P<0.001).

FIGS. 4A-4C show the effect of FATS gene defect on NK cells in the spleens of mice with melanoma. B16 cells were injected subcutaneously into the WT and KO mice for a tumor-graft experiment and the mice were sacrificed 20 after days. Mononuclear cells were separated from the spleens of the WT and KO mice and then analyzed by flow cytometry for detecting the frequency and activation of NK cells. FIG. 4A is a typical flow cytometry plot showing the frequency of NK cells in the spleens from the WT and KO mice with melanoma. FIG. 4B is a statistical chart showing the frequency of NK cells in the spleens from the WT and KO mice with melanoma. FIG. 4C includes a typical flow cytometry plot (left) and a statistical chart (right) showing the activation of NK cells, and the vertical axis indicates the mean fluorescence intensity (MFI) (*, P<0.05; **, P<0.01; ***, P<0.001).

FIGS. 5A-5D show that FATS gene defect increases the frequency and activation level of CTLs in the spleens of mice with melanoma. B16 cells were injected subcutaneously into the WT and KO mice for a tumor-graft experiment and the mice were sacrificed after 20 days. Mononuclear cells were separated from the spleens of the WT and KO mice and analyzed by flow cytometry for detecting the frequency and activation of CTLs.

FIG. 5A is a typical flow cytometry plot showing the frequency of CTLs in the spleens from the WT and KO mice with melanoma. FIG. 5B is a statistical chart showing the frequency of CTLs in the spleens from the WT and KO mice with melanoma. FIG. 5C is a typical flow cytometry plot showing the activation of CTLs.

FIG. 5D is a statistical chart showing the frequency of highly positive CD44⁺ in highly activated CTLs (*, P<0.05; **, P<0.01; ***, P<0.001).

FIGS. 6A-6B show that FATS gene defect does not change the frequency of IFN-γ⁺ CTLs and Th1 cells in the spleens of mice with melanoma. B16 cells were injected subcutaneously into the WT and KO mice for a tumor-graft experiment and the mice were sacrificed after 20 days. Mononuclear cells were separated from the spleens of the WT and KO mice and analyzed by flow cytometry for detecting the frequency of IFN-γ⁺ CTLs and Th1 cells. FIG. 6A includes a typical flow cytometry plot (left) and a statistical chart (right) showing the frequency of Th1 cells in the spleens from the WT and KO mice with melanoma. FIG. 6B includes a typical flow cytometry plot (left) and a statistical chart (right) showing the frequency of IFN-γ⁺ CTLs in the spleens from the WT and KO mice with melanoma.

FIGS. 7A-7C show the effect of FATS gene defect on Tregs (regulatory T cells) and MDSCs (myeloid-derived suppressor cells) in the spleens of mice with melanoma. B16 cells were injected subcutaneously into the WT and KO mice for a tumor-graft experiment and the mice were sacrificed after 20 days. Mononuclear cells were separated from the spleens of the WT and KO mice and then analyzed by flow cytometry for detecting the changes in immune cell subsets (Tregs and MDSCs).

FIG. 7A is a typical flow cytometry plot showing the frequency of Tregs in the spleens from the WT and KO mice with melanoma. FIG. 7B is a statistical chart showing the frequency of Tregs in the spleens from the WT and KO mice. FIG. 7C is a statistical chart showing the frequency of MDSCs in the spleens from the WT and KO mice (*, P<0.05; **, P<0.01; ***, P<0.001).

FIG. 8 shows that FATS gene defect increases the expression of T cell-activating factors in serum of mice with melanoma. B16 cells were injected subcutaneously into the WT and KO mice for a tumor-graft experiment. Blood samples were extracted from the eyeballs of the WT and KO mice. Sodium heparin was added to the plasma which was then centrifuged. Serum was extracted for a multi-cytokine ELISA assay (Bio-Plex). The figure includes a statistical chart showing the expression level of IL-2, IFN-γ, IL-12, IL-1β, TNF-α and IL-10 in the serum of the WT and KO mice (*, P<0.05; **, P<0.01; ***, P<0.001).

FIGS. 9A-9D show that FATS gene defect increases T cells frequency in a tumor immune microenvironment in mice with melanoma. B16 cells were injected subcutaneously into the WT and KO mice for a tumor-graft experiment. The mice were sacrificed after 20 days. Mononuclear cells were separated from the tumor tissue of the WT and KO mice, and were analyzed by flow cytometry for detecting the changes in the frequency of total T cells and CTLs. FIG. 9A is a typical flow cytometry plot showing the frequency of total T cells in the tumor immune microenvironment of the WT and KO mice with melanoma. FIG. 9B is a statistical chart showing the frequency of total T cells in the tumor immune microenvironment of the WT and KO mice with melanoma. FIG. 9C is a typical flow cytometry plot showing the frequency of CTLs in the tumor immune microenvironment of the WT and KO mice with melanoma. FIG. 9D is a statistical chart showing the frequency of CTLs in TME in the WT and KO mice with melanoma (*, P<0.05; **, P<0.01; ***, P<0.001).

FIGS. 10A-10D show that FATS gene defect increases NK and γδ T cells frequency in tumor immune microenvironment in the mice with melanoma. B16 cells were injected subcutaneously into the WT and KO mice for a tumor-graft experiment. The mice were sacrificed 20 days later. Mononuclear cells were separated from the tumor tissue of the WT and KO mice, and were analyzed by flow cytometry for detecting the changes in immune cell subsets (NK and γδ T cells). FIG. 10A is a typical flow cytometry plot showing the frequency of NK cells in the tumor immune microenvironment of the WT and KO mice with melanoma. FIG. 10B is a statistical chart showing the frequency of NK cells in the tumor immune microenvironment of the WT and KO mice with melanoma. FIG. 10C is a typical flow cytometry plot showing the frequency of γδ T cells in the tumor immune microenvironment of the WT and KO mice with melanoma. FIG. 10D is a statistical chart showing the frequency of γδ T cells in the tumor immune microenvironment of the WT and KO mice with melanoma (*, P<0.05; **, P<0.01; ***, P<0.001).

FIGS. 11A-11B show that FATS gene defect enhances the activation of CTLs in a melanoma tumor microenvironment. B16 cells were injected subcutaneously into the WT and KO mice for a tumor-graft experiment. The mice were sacrificed after 20 days. Mononuclear cells were separated from the tumor tissue of the WT and KO mice, and were analyzed by flow cytometry for detecting the activation level of CTLs. FIG. 11A is a typical flow cytometry plot showing the frequency of activated CTLs in the tumor immune microenvironment of the WT and KO mice. FIG. 11B is a statistical chart showing the frequency of activated CTLs in the tumor immune microenvironment of the WT and KO mice (*, P<0.05; **, P<0.01; ***, P<0.001).

FIGS. 12A-12B show that FATS gene defect increases the frequency of IFN-γ⁺ CTLs and Th1 cells in the tumor immune microenvironment. B16 cells were injected subcutaneously into the WT and KO mice for a tumor-graft experiment. The mice were sacrificed after 20 days. Mononuclear cells were separated from the tumor tissue of the WT and KO mice, and were analyzed by flow cytometry for detecting the frequency of IFN-γ⁺ CTLs and Th1 cells. FIG. 12A includes a typical flow cytometry plot (left) and a statistical chart (right) showing the frequency of IFN-γ⁺ CTLs in the tumor immune microenvironment of the WT and KO mice. FIG. 12B includes a typical flow cytometry plot (left) and a statistical graph (right) showing the frequency of Th1 cells in the tumor immune microenvironment of the WT and KO mice (*, P<0.05; **, P<0.01; ***, P<0.001).

FIGS. 13A-13C show the effect of FATS gene defect on Tregs and MDSCs in the tumor immune microenvironment of mice with melanoma. B16 cells were injected subcutaneously into the WT and KO mice for a tumor-graft experiment. The mice were sacrificed after 20 days. Mononuclear cells were separated from the tumor tissue of the WT and KO mice, and were analyzed by flow cytometry for detecting the changes in immune cell subsets (Tregs and MDSCs). FIG. 13A is a typical flow cytometry plot showing Tregs frequency in the tumor microenvironment of the WT and KO mice with melanoma. FIG. 13B is a statistical chart showing Tregs frequency in the tumor immune microenvironment of the WT and KO mice with melanoma. FIG. 13C is a statistical chart showing MDSCs frequency in the tumor immune microenvironment of the WT and KO mice with melanoma (*, P<0.05; **, P<0.01; ***, P<0.001).

FIGS. 14A-14E show that FATS gene defect promotes M1 type macrophages and inhibits M2 type macrophages in the tumor immune microenvironment. B16 cells were injected subcutaneously into the WT and KO mice for a tumor-graft experiment. The mice were sacrificed after 20 days. A Part of the tumor tissue was fixed in methanol and sectioned for detecting the expression of CD206 by immunofluorescence assay. Mononuclear cells were separated from the other part of the tumor tissue and analyzed by flow cytometry for detecting immune cell subsets (M1 and M2 type macrophages). FIG. 14A is a typical flow cytometry plot showing the frequency of M1 type macrophages in the tumor immune microenvironment of the WT and KO mice with melanoma. FIG. 14B is a statistical chart showing the frequency of M1 type macrophages in the tumor immune microenvironment of the WT and KO mice with melanoma. FIG. 14C is a typical flow cytometry plot showing the frequency of M2 type macrophages in the tumor immune microenvironment of the WT and KO mice with melanoma. FIG. 14D is a statistical chart showing the frequency of M2 type macrophages in the tumor immune microenvironment of the WT and KO mice with melanoma. And FIG. 14E is an immunofluorescence image indicating the expression level of CD206 in the tumor tissue sections (by showing cells with CD206 stained) (*, P<0.05; **, P<0.01; ***, P<0.001).

FIGS. 15A-15B show that FATS gene defect increases the gene expression of M1 type macrophage-associated factors, and inhibits the gene expression of M2 type macrophage-associated factors. B16 cells were injected subcutaneously into the WT and KO mice for a tumor-graft experiment. The mice were sacrificed after 20 days. Mononuclear cells were separated from the tumor tissue of the WT and KO mice, and were sorted by flow cytometry to obtain macrophages. RNA was extracted from the macrophages for detecting the expression level (transcriptional level) of M1 and M2 type macrophage polarization-associated factors. FIG. 15A shows the gene expression level (mRNA level) of M1 type macrophage-associated factors (IL-12, TNFα and NOS2) expressed by macrophages in the tumor immune microenvironment of the WT and KO mice. FIG. 15B shows the gene expression level (mRNA level) of M2 type macrophage-associated factors (IL-10, Agr1, Mrc1 (CD206) and CCL22) expressed by macrophages in the tumor immune microenvironment of the WT and KO mice (*, P<0.05; **, P<0.01; ***, P<0.001).

FIG. 16 shows that FATS gene defect inhibits angiogenesis in a melanoma tumor microenvironment. B16 cells were injected subcutaneously into the WT and KO mice for a tumor-graft experiment. The mice were sacrificed 20 days later and tumor tissue from the mice was fixed in methanal and sectioned for detecting the expression of CD31 in the tumor tissue by immunofluorescence assay. The images show the expression of CD31 in tumor tissue from the WT (left) and KO (right) mice, where the arrows indicate the cells with CD31 stained, with background stained by DAPI.

FIGS. 17A-17B show that FATS gene defect enhances the presenting ability of macrophages in a melanoma tumor microenvironment. B16 cells were injected subcutaneously into the WT and KO mice for a tumor-graft experiment. The mice were sacrificed after 20 days. Mononuclear cells were separated from the tumor tissue of the WT and KO mice, and sorted by flow cytometry to obtain macrophages. The macrophages were treated with mitomycin C, and mixed with CD3⁺ T cells obtained by magnetic beads sorting (MACs) in a ratio of 1:4 for a co-culture. A flow cytometry assay was performed to detect the changes in T cells proliferation. FIG. 17A is a statistical chart showing the proliferation index of the co-cultured T cells. FIG. 17B shows the expression level of IL-2 (*, P<0.05; **, P<0.01; ***, P<0.001).

FIGS. 18A-18C show that macrophages in tumor of FATS gene defect mice have stronger cell-killing ability. B16 cells were injected subcutaneously into the WT and KO mice for a tumor-graft experiment. The mice were sacrificed after 20 days. Mononuclear cells were separated from the tumor tissue of the WT and KO mice, and sorted by flow cytometry to obtain macrophages. The macrophages were mixed with B16 cells in a ratio of 40:1 for a co-culture. A flow cytometry assay was performed 1 day later to analyze the apoptosis of the B16 cells. FIG. 18A is a typical flow cytometry plot showing the apoptosis of B16 cells after the co-culture. FIG. 18B is a statistical chart showing the level of early apoptotic B16 cells. FIG. 18C shows the expression level (concentration) of NO in the culture supernatant of the co-culture (*, P<0.05; **, P<0.01; ***, P<0.001).

FIGS. 19A-19D show that FATS defect promotes the polarization of macrophages to M1 type macrophages and inhibits the polarization of macrophages to M2 type macrophages. Bone marrow cells were separated from the WT and KO mice, and added with M-CFS for directed differentiation into M0 type macrophages. The M0 type macrophages were then added with IFN-γ and LPS, or IL-4 to polarize into M1 or M2 type macrophages, and the macrophages were collected 16 hours later, and were analyzed by flow cytometry for detecting the frequency of M1 and M2 type macrophages. FIG. 19A is a typical flow cytometry plot showing the frequency of M1 type macrophages after the polarization of M0 type macrophages into M1 type macrophages. FIG. 19B is a statistical chart showing the frequency of M1 type macrophages after the polarization. FIG. 19C is a typical flow cytometry plot showing the frequency of M2 type macrophages after the polarization of M0 type macrophages into M2 type macrophages. FIG. 19D is a statistical chart showing the frequency of M2 type macrophages after the polarization (*, P<0.05; **, P<0.01; ***, P<0.001).

FIG. 20 shows that FATS gene defect promotes the gene expression of IL-12, TNF-α and NOS2 in M1 type macrophages. Bone marrow cells were separated from the WT and KO mice, and added with M-CFS for directed differentiation into M0 type macrophages. The M0 type macrophages were then added with IFN-γ and LPS to polarize into M1 type macrophages. The macrophages were collected to detect the expression of M1 type macrophage-associated factors with real-time quantitative PCR. The figure is a statistical chart showing the gene expression (mRNA) level of TNF-α, NOS2 and IL-12 in M1 type macrophages (*, P<0.05; **, P<0.01; ***, P<0.001).

FIG. 21 shows that FATS gene defect inhibits the gene expression of Arg1, Mrc1, Retnla and CCL22 in M2 type macrophages. Bone marrow cells were separated from the WT and KO mice, and added with M-CFS for directed differentiation into M0 type macrophages. The M0 type macrophages were then added with IL-4 to polarize into M2 type macrophages. The macrophages were collected to detect the expression of M2 type macrophage-associated factors with real-time quantitative PCR. This figure is a statistical chart showing the gene expression level (mRNA) of Arg1, Mrc1, Retnla and CCL22 in M2 type macrophages (*, P<0.05; **, P<0.01; ***, P<0.001).

FIGS. 22A-22C show that FATS gene defect promotes the apoptosis of M2 type macrophages. Bone marrow cells were separated from the WT and KO mice, and added with M-CFS for directed differentiation into M0 type macrophages. The M0 type macrophages were then added with IL-4 to polarize into M2 type macrophages. The macrophages were collected for an apoptosis kit assay to detect apoptosis of M2 type macrophages and the expression of apoptosis-associated proteins by immunoblot. FIG. 22A is a typical flow cytometry showing the frequency of early apoptotic M2 type macrophages. FIG. 22B is a statistical chart showing the frequency of early apoptotic M2 type macrophages. FIG. 22C shows the expression level (protein) of Cleaved-caspase3 and Bcl2 (both as apoptosis signal) in M2 type macrophages (*, P<0.05; **, P<0.01; ***, P<0.001).

FIG. 23 shows that FATS gene defect activates NF-κB signaling pathway in macrophages. Bone marrow cells were separated from the WT and KO mice, and were added with M-CFS for directed differentiation into M0 type macrophages. The macrophages obtained were stimulated with LPS to extract protein for a phosphorylation level detection of intracellular NF-κB signaling pathway at different time by immunoblot, indicating the activation level of the NF-κB signaling pathway.

FIGS. 24A-24C show that an adoptive therapy using bone marrow derived macrophages (BMDMs) with FATS gene defect can be performed to significantly inhibit tumor growth. Ten female C57BL/6 mice, aged 6-8 weeks and weighing 18-20 g, were subcutaneously injected with B16 cells (2×10⁵/mouse) to build a mouse subcutaneous melanoma xenograft model. The mice were randomly divided into two groups (5 mice per group). M0 type macrophages, derived from the directed differentiation of bone marrow cells (stimulated with LPS for 12 hours) of the WT and KO mice, were adoptively infused into the C57BL/6 mice on the 2nd and 7th day of tumor-graft experiment, respectively, and tumor size was continuously monitored. The C57BL/6 mice were sacrificed after 16 days of tumor-graft, and tumor tissue was separated for weighing and imaging.

FIG. 24A shows a growth curve of melanoma after the adoptive infusion of BMDMs of the WT and KO mice into the C57BL/6 mice. FIG. 24B is an image showing a representative result of the melanoma size after the adoptive infusion of BMDMs of the WT and KO mice into the C57BL/6 mice. FIG. 24C shows a final weight of tumor after the adoptive infusion of BMDMs of the WT and KO mice into the C57BL/6 mice (*, P<0.05; **, P<0.01; ***, P<0.001).

FIGS. 25A-25B show that an adoptive infusion of BMDMs transfected with FATS-siRNA can be performed to significantly inhibit tumor growth. Bone marrow cells were separated from WT mice, and added with M-CFS for directed differentiation into M0 type macrophages. The M0 type macrophages were then transfected with FATS-siRNA and NC-siRNA, respectively. 24 hours later, the transfected macrophages were stimulated with LPS (1 μg/ml) for 12 hours. The stimulated cells were collected, with cell concentration adjusted to 1×10⁷/ml, and were introduced into mice by a tail vein injection (100 μl per mouse). Such adoptive infusion of the transfected macrophages was performed on the 2nd and 7th day of tumor-graft, and tumor growth was continuously monitored. FIG. 25A shows the expression level (mRNA) of FATS gene in macrophages detected by RT-PCR after a 24-hour transfection with FATS/NC-siRNA. FIG. 25B shows a growth curve of melanoma in the WT and KO mice with siRNA infusion, respectively (*, P<0.05; **, P<0.01; ***, P<0.001).

FIGS. 26A-26B show that knockout of FATS gene inhibits the genesis and progression of a subcutaneous xenograft of H7 pancreatic cancer. 1×10⁶ of H7 pancreatic cancer cells were subcutaneously injected into the right back of mice (WT mice, n=7; KO mice, n=8), and tumor size was observed and measured till the 30th day of the tumor-graft experiment. The mice were sacrificed to measure the volume and weight of the subcutaneous xenograft. FIG. 26A shows a growth curve of the subcutaneous xenograft of H7 pancreatic cancer in the WT and KO mice. FIG. 26B is an image showing a representative result of tumor size of the WT and KO mice. FIG. 26C shows a final weight of tumor from the WT and KO mice (*, P<0.05; **, P<0.01; ***, P<0.001).

FIG. 27 shows that knockout of FATS gene prolongs the survival of subcutaneous H7 cells tumor-graft mice. 1×10⁶ of H7 cells were subcutaneously injected into the right back of mice (WT mice, n=7; KO mice, n=7) followed by an observation every other day for survival status. The mice were sacrificed on the 50th day of the tumor-graft experiment, and the survival rate of the mice was calculated (*, P<0.05; **, P<0.01; ***, P<0.001).

FIG. 28 shows the frequency of CD4⁺ T lymphocytes, CD8⁺ T lymphocytes and Tregs, as well as the frequency of CD44hi (in CD8+ lymphocytes) in spleens of WT and KO mice subcutaneously injected with H7 cells for tumor-graft. H7 cells were subcutaneously injected into the WT and KO mice for a tumor-graft experiment. The mice were sacrificed after 30 days. Mononuclear cells were separated from the spleens of the WT and KO mice, and were analyzed by flow cytometry (*, P<0.05; **, P<0.01; ***, P<0.001).

FIG. 29 shows that the knockout of FATS gene increases the frequency of total T lymphocytes, CD4⁺ T lymphocytes and CD8⁺ T lymphocytes in tumor from mice with pancreatic cancer. H7 cells were subcutaneously injected into the WT and KO mice for a tumor-graft experiment. The mice were sacrificed after 30 days. Tumor-infiltrating mononuclear cells were separated from the tumor tissue of the WT and KO mice, and were analyzed by flow cytometry for detecting the changes in immune cell subsets (total T lymphocytes, CD4⁺ T lymphocytes, CD8+T lymphocytes and Tregs) (*, P<0.05; **, P<0.01; ***, P<0.001).

### DETAILED DESCRIPTION OF THE PRESENT INVENTION

This application will be further described below in conjunction with embodiments.

The terminology used in the specification and claims has the general definitions as described below, unless otherwise specified. These definitions are intended to have the meanings as appreciated by those skilled in the art.

Term “conservative” means that the amino acid sequence or nucleic acid sequence is highly similar or identical to an original sequence, and has the ability to preserve the basic structure, biological activity or function of the original sequence. Generally, these sequences can be obtained through substitution of a similar amino acid residue or a allele (synonymous codon).

Term “variant” includes an amino acid sequence or nucleic acid sequence with one or more amino acid or nucleotide modifications, including insertions, deletions or substitutions of amino acids or nucleotides in an amino acid sequence or nucleic acid sequence. Variants may have conservative modifications where the substituted amino acid has similar structural or chemical properties to the original amino acid, such as substitution between leucine and isoleucine. Variants may also have non-conservative modifications.

Term “homologous” includes completely homologous and partially homologous. “Homologous”, in terms of polypeptide, protein or amino acid sequence, means that they contain similar amino acid sequences with same or similar structure or function. “Homologous”, in terms of nucleic acid sequences, means that they contain a similar or complementary nucleic acid sequences, as well as nucleic acid sequences according to the codon preference of organism of the construct. “Homologous” has a relatively broad meaning herein, for example, includes sequences having a certain percentage of identity (amino acid sequence or nucleic acid sequence), or variants of the original sequence.

Term “derivative”, in terms of polypeptide, protein or amino acid sequence, refers to a related polypeptide, protein or amino acid sequence derived from the original polypeptide, protein or amino acid sequence with similar property, activity or function. For example, the polypeptide, protein or amino acid sequence used herein includes a derivative which may be obtained via (i) fusion of a mature polypeptide with a compound; or (ii) fusion or insertion of an additional amino acid sequence (linker, protein purification label sequence, restriction site, etc.) into an amino acid sequence, and other means. “Derivative”, in terms of nucleic acid sequence, refers to a subsequent related sequence derived from the original sequence with similar property, activity or function, which may be obtained via (i) continuous or intermittent insertion, deletion or substitution of one or more bases (preferably, a substitution of an allele) where insertion, deletion and/or substitution of one of more amino acid residues may be present in a sequence or gene; (ii) modifications of one or more bases in a sequence or gene; or (iii) fusion or insertion of a gene encoding an additional amino acid sequence into a sequence or gene, and other means.

Term “inhibitor” includes antagonists, down-regulators, retarder, blockers, nucleic acid inhibitors, and the like.

Term “down-regulation” refers to reduction of the activity, stability and effective action time of FATS gene or expression product thereof, inhibition of the transcription and/or translation of FATS gene, reduction of the expression of FATS gene expression product and etc.

Term “interfering molecule” generally refers to a substance capable of down-regulating FATS gene or expression product thereof, including a small interfering RNA, dsRNA, shRNA, microRNA and antisense nucleic acid.

Designing an interfering molecule according to particular target sequences is known to those skilled in the art and achievable. Such interfering molecule can also be delivered into body by a variety of means known in the art (e.g., by adopting appropriate reagents) to exert its effect of down-regulating FATS gene or expression product thereof.

According to the correlation between FATS gene or its expression product and melanoma, a functional product capable of acting on, and in particular, down-regulating FATS gene or its expression product can be screened. Various methods for screening the functional product are known in the art.

The correlation between FATS gene or its expression product and melanoma was illustrated in detail with reference to particular experiments and analysis.

### Example 1 Regulation of FATS Gene Defect on Melanoma and Tumor Immune Microenvironment in Mice

### 1.1 Subjects and Methods

### 1.1.1 Materials, Reagents and Instruments

### 1.1.1.1 Reagents

| | |
|---|---|
| Collagenase IV | Sigma-Aldrich, US |
| Mouse Lymphocyte Separating Medium | TBD Science, Tianjin |
| Triple Stimulant | BioLegend, US |
| Bovine Serum Albumin (BSA) | GIBCO, US |
| Permeabilization Wash Buffer | eBioscience, US |
| Cytofix/Cytoperm Buffer | eBioscience, US |
| Fixation Buffer | eBioscience, US |
| CFSE | Invitrogen, US |
| M-MLV Reverse Transcriptase | Invitrogen, US |
| Real-time PCR Kit | DBI ® Bioscience Co., Ltd. |

### 1.1.1.2 Cell Lines

B16 cells.

### 1.1.1.3 Antibodies

Anti-mouse CD3-PE, Anti-mouse CD8a-APC, Anti-mouse NK1.1-APC, Anti-mouse γδ-APC, Anti-mouse CD3-PE-CY7, Anti-mouse CD4-APC, Anti-mouse Ly6C-PE, Anti-mouse Ly6G-PeCy5.5, Anti-mouse CD44-PE, Anti-mouse IFN-γ-PE, Anti-mouse CD11b-FITC, Anti-mouse CD11b-PE and Anti-mouse MHC-II-PE, eBioscience, US; Anti-mouse CD25-FITC, Anti-mouse Foxp3-PE, Anti-mouse F4/80-APC, Anti-mouse CD206-FI FITC, Biolegend, US.

### 1.1.1.4 Primers

| | Forward primers | Reverse primers |
|---|---|---|
| TNF-α | GAGGCCAAGCCCTGGTATG | CGGGCCGATTGATCTCAGC |
| NOS2 | GTTCTCAGCCCAACAATACAAGA | GTGGACGGGTCGATGTCAC |
| IL-12 | AAAAGGAGGCGAGGTTCTAA | CCCTTGGGGGTCAGAAGAG |
| IL-1β | GAAATGCCACCTTTTGACAGTG | TGGATGCTCTCATCAGGACAG |
| Arg1 | CTCCAAGCCAAAGTCCTTAGAG | GGAGCTGTCATTAGGGACATCA |
| CCL22 | CTCTGCCATCACGTTTAGTGAA | GACGGTTATCAAAACAACGCC |
| Mrc1 | CTCTGTTCAGCTATTGGAGC | TGGCACTCCCAAACATAATTTGA |
| Rein1a | CCAATCCAGCTAACTATCCCTCC | ACCCAGTAGCAGTCATCCCA |
| TGF-β | CCACCTGCAAGACCATCGAC | CTGGCGAGCCTTAGTTTGGAC |
| GAPDH | AGGTCGGTGTGAACGGATTTG | GGGGTCGTTGATGGCAACA |

### 1.1.2 Preparation of Reagents

### 1.1.2.1 0.01 M PBS:

Na₂HPO₄ (1.54 g), KH₂PO₄ (0.2 g), NaCl (8.0 g) and KCl (0.2 g) were dissolved completely in ultra-pure water and was diluted to 100 ml to obtain a diluted solution with pH of 7.2-7.4. The diluted solution was sterilized at high pressure to produce 0.01 M PBS which was stored at 4° C. for use.

### 1.1.2.2 Flow Cytometry Staining Buffer (SB)

10 ml of FBS and 5 ml of 10% NaN₃ were added to 500 ml of PBS and mixed to obtain a staining buffer which was stored at 4° C. for use.

### 1.1.2.3 Antibody Diluents

0.2 mg of BSA and 1 ml of 10% NaN₃ were added to 100 ml of PBS under stirring to obtain an antibody diluent which was stored at 4° C. for use.

### 1.1.2.4 4% Paraformaldehyde

2 g of paraformaldehyde was added to 45 ml of ultra-pure water to which NaOH of 1 mol/L was then added so as to obtain a mixture. The mixture was stored at 56° C. overnight to allow complete dissolution of paraformaldehyde, and was cooled to room temperature. 5 ml of 10×PBS, 50 μl of 1 mol/L CaCl₂ and 50 μl of 1 mol/L MgCl₂ were introduced to the mixture with pH of 7.3 so as to produce 4% paraformaldehyde. The 4% paraformaldehyde was stored at 4° C. in the dark for use.

### 1.1.2.5 Flow Cytometry Staining Blocking Buffer

800 μl of 10% BSA was mixed with 200 μl of rat serum uniformly to produce a blocking buffer which was stored at 4° C. in the dark for use.

### 1.1.2.6 Buffer for Cell Sorting (0.5% BSA/PBS)

10% BSA was prepared. The 10% BSA was diluted 20 times with 1×PBS to obtain 0.5% BSA/PBS which was stored at 4° C. for use.

### 1.1.2.7 Heat Induced Antigen Retrieval Solution

41 ml of sodium citrate and 9 ml of citric acid were added to 450 ml of ultra-pure water to mix uniformly so as to obtain a heat induced antigen retrieval solution which was stored at room temperature for use.

### 1.1.3 Experiments

### 1.1.3.1 Establishment of Mouse Melanoma Model

### 1.1.3.1.1 Animals

1) Mice with FATS gene defect (C57BL/6 mice) were provided by Professor Li Zheng from Tianjin Cancer Hospital, and were fed in the Experimental Animal Center of Tianjin Medical University. Mice room, SPF, with ambient temperature of 20-25° C. and RH (relative humidity) of 40-60%. Knockout FATS gene in the mice is represented by SEQ ID NO: 1.

2) Wild type (WT) mice were specific pathogen free (SPF) C57BL/6 mice, 8 weeks of age, weighing about 20 g, and were provided by Beijing Vital River Laboratory Animal Technology Co., Ltd. The mice were fed in the Experimental Animal Center of Tianjin Medical University. Mice room, SPF, with ambient temperature of 20-25° C. and RH of 40-60%.

### 1.1.3.1.2 Establishment of Ectopic Mouse Melanoma Model

B16 cells were cultured in DMEM medium containing 10% FBS and 1% double antibody to logarithmic growth phase. The cells were trypsinized and collected for a centrifugation and then the trypsinized cells were washed twice with PBS for counting. The trypsinized cell concentration was adjusted to 2×10⁶/ml. Each mouse was injected with 100 μl (31 mice in total) of the trypsinized cell, i.e. 2×10⁵ cells per mouse. The tumor growth of the mice was observed every other day, and the length and width of the tumor were measured and recorded with a vernier caliper. the mice were sacrificed by cervical vertebra luxation after anesthesia after 20 days of the tumor implantation, and spleens and tumor tissue were separated, weighed and imaged. The tumor length and width were further measured to calculate the tumor volume according to a formula: V (volume)=(length×width²)/2 (mm³).

1.1.3.2 Separation of Mononuclear Cells from Spleen and Tumor Tissue
1.1.3.2.1 Separation of Mononuclear Cells from Spleen

Spleen tissue was transferred from the mice to a clean bench. The spleen tissue was placed in a disposable cell sieve, and cut with scissors, and was then ground into a single cell suspension with a 1 ml syringe needle. The cell suspension was collected to a 15 ml sterile centrifuge tube, and was then centrifuged at 1300 rpm for 5 minutes. The supernatant was discarded, and the cells were resuspended with 900 μl of ultra-pure water, and then quickly added with 100 μl of 10×PBS to obtain a cell solution. Lysed red blood cell mass appeared in the cell solution and was picked out. The centrifuge tube containing the cell solution was filled up with serum-free 1640 medium, and then subjected to a centrifugation at 1300 rpm for 5 minutes. The supernatant was discarded, and the cells were resuspended in 1640 medium containing 10% FBS and 1% double antibody, and cultured for use.

1.1.3.2.2 Separation of Mononuclear Cells from Tumor Tissue

Tumor tissue was transferred from mice to a clean bench. The tumor tissue was cut with a flat-head shear to small pieces of about 1 mm in diameter followed by adding with about 10 ml of digestive enzyme (0.05 mg/ml collagenase IV+0.05 mg/ml hyaluronidase+0.05 mg/ml DNase I) for a 1-hour digestion at 37° C. to produce a digested product. The digested product was transferred to a disposable cell sieve, and ground with the needle head of a 1 ml sterile syringe to obtain a ground fluid. Meanwhile, 1×PBS was added to filter the ground fluid and the filtrate was collected in a 15 ml sterile centrifuge tube, and centrifuged at 1500 rpm for 5 min. The centrifugation was repeated twice. The supernatant was discarded, and the cells were resuspended with 4 ml of sample dilution followed by adding with an equal volume of mouse lymphocyte separatory solution to obtain a cell solution. The cell solution was centrifuged at 2000 rpm for 20 mins at room temperature, with the speed block and brake block set at 0. The centrifuge tube was transferred smoothly, and liquid above a volume of 0.5 ml above the white film-layer was sucked out with a 5 ml pipette. Then the white-film layer was sucked out with a 200 μl pipette and placed in a 15 ml sterile centrifuge tube and then 1×PBS was added at a volume ratio of 3:1. The centrifuge tube was centrifuged 3 times at 2000, 1800 and 1500 rpm, respectively and each time for 5 minutes. The supernatant was discarded, and the cells were resuspended with 1640 medium containing 10% FBS and 1% double antibody and cultured for use.

### 1.1.3.3 Flow Cytometry Detection of Immune Cell Subsets

Cells to be tested were collected, and were centrifuged at 1500 rpm for 5 minutes. The supernatant was discarded and the cells were resuspended with 1 ml of 1×PBS followed by another centrifugation at 1500 rpm for 5 minutes. The supernatant was discarded and the cells were divided and added to a blank tube, a single-staining tube and an isotype control tube. Each tube together with a detector tube was added with 1×PBS to a volume of about 100 μl.

Blocking: each tube was added with 25 μl of antibody blocking buffer for flow cytometry detection, and kept in the dark at 4° C. for 30 minutes.

Surface staining: each tube was added with a fluorescence-labeled rat anti-mouse antibody with the isotype control tube further added with a matching isotype antibody according to the experimental design and requirements.

Avoiding false positive: the tubes were kept in the dark at 4° C. for 30 minutes. Each tube was added with 1 ml of PBS for resuspension, and then centrifuged at 1500 rpm for 5 minutes and washed twice. The supernatant was discarded and each tube was added with 50-100 μl of 4% paraformaldehyde or fixing buffer for cell fixation (can be stored for a short time at 4° C.). The fixed cells were detected by flow cytometry.

The immune cell subsets to be detected are:
\textcircled{1} total T cells: CD3-PE;
\textcircled{2} cytotoxic T lymphocytes (CTL): CD3-PE/CD8a-APC;
\textcircled{3} γδ T cells: CD3-PE/γδ-APC;
\textcircled{4} natural killer T cells (NKT): CD3-PE/NK1.1;
\textcircled{5} myeloid-derived suppressor cells (MDSC): CD11b-FITC/Ly6C-PE/Ly6G-PeCy 7;
\textcircled{6} M1 type macrophages: CD11b-FITC/MHC-II-PE/F4/80-APC CD11b-FITC/CD11c-PE/F4/80-APC;
\textcircled{7} M2 type macrophages: CD206-FITC/CD11b-PE/F4/80-APC;
\textcircled{8} activated cytotoxic T lymphocytes: CD3-FITC/CD44-PE/CD8-APC;
\textcircled{9} activated helper T cells 1 (Th1): CD3-FITC/CD44-PE/CD4-APC.

### 1.1.3.4 Flow Cytometry Detection of Tregs (Regulatory T Cells)

Spleen or tumor cells were collected and divided to a blank tube, a single-staining tube, an isotype test tube and an experimental tube.

Surface staining: CD25-FITC/CD3-Pe-Cy 7/CD4-APC was incubated in the dark at 4° C. for 30 minutes. Each tube was added with 1 ml of staining buffer SB, and centrifuged twice at 1500 rpm for 5 minutes (SB added with FBS in PBS was used to protect the cells from being damaged by intracellular staining). The supernatant was discarded, and each tube was added with 250 μl of Foxp3 Cytofix/Cytoperm buffer, and covered with light-proof film and kept at 4° C. for 15-20 minutes. Each tube was added with 1-2 ml of permeabilization wash buffer (the commercial 10×buffer was diluted to 1× with ultra-pure water before use), and was centrifuged at 1800 rpm for 7 minutes.

Blocking: each tube was added with 25 μl of blocking buffer, and was kept at 4° C. for 30 minutes in the dark. Each tube was added with rat anti-mouse fluorescent antibody Foxp3-PE, and the isotype tube was added with a matching isotype antibody according to the instruction, and the tubes were kept in the dark for 30 minutes at room temperature. Each tube was added with 1-2 ml of permeabilization wash buffer, and was centrifuged at 1800 rpm for 7 minutes. The supernatant was discarded, and each tube was added with 1 ml of SB followed by a centrifugation at 1800 rpm for 7 minutes with the supernatant discarded. Each tube was then added with 50-100 μl of 4% paraformaldehyde or fixing buffer for fixation (can be stored for a short time at 4° C.) and for further flow cytometry detection.

### 1.1.3.5 Flow Cytometry Detection of Intracellular Factors of Cytotoxic T Lymphocytes

Triplet stimulant (including PMA, calcium ionomycin and BFA) was added to the cells to be tested. The cells were placed in a CO2 cell incubator for a stimulation for 4-5 hours. The cells were collected and centrifuged at 1500 rpm for 5 minutes. The supernatant was discarded and the cells were resuspended with 1 ml of 1×PBS and centrifuged at 1500 rpm for 5 minutes. The cells were divided to a blank tube, a single-staining tube, an isotype test tube and an experimental tube.

Surface staining: the tubes were incubated in the dark at 4° C. for 30 minutes. Each tube was added with 1 ml of staining buffer SB, and centrifuged twice at 1500 rpm for 5 minutes. The supernatant was discarded, and each tube was added with 250 μl of 4% PFA or fixing buffer for fixation which was kept in the dark at 4° C. for 30 minutes or overnight.

Fixation: the tubes were centrifuged and the supernatant was discarded. Each tube was added with 1 ml of 1×permeabilization wash buffer, and was centrifuged at 1800 rpm for 7 minutes. The supernatant was discarded, and then each tube was added with 250 μl of 1× permeabilization wash buffer, and was kept at 4° C. in the dark for 15-20 minutes. Each tube was added with 1 ml of permeabilization wash buffer, and was centrifuged at 1800 rpm for 7 minutes with the supernatant discarded.

Blocking: each tube was added with 25 μl of blocking buffer, and was kept in the dark at 4° C. for 30 minutes. Each tube was added with rat anti-mouse fluorescent antibody IFN-γ-PE, and the isotype tube was further added a matching isotype antibody according to the instruction, and all tubes were kept in the dark at 4° C. for 30 minutes. Each tube was added with 1-2 ml of permeabilization wash buffer, and centrifuged at 1800 rpm for 7 minutes with the supernatant discarded. And then each tube was added with 1 ml of SB, and centrifuged at 1800 rpm for 7 minutes. Next, each tube was added with 50-100 μl of 4% paraformaldehyde or fixing buffer for fixation (can be stored for a short time at 4° C.) and for further flow cytometry detection.

### 1.1.3.6 RNA Extraction

Cells added with Trizol frozen at −80° C. were transferred and thawed at room temperature, and vortexed for 15 s for a complete mix followed by a standing for 10 minutes at room temperature. Each tube was added with 200 μl of chloroform, and vortexed followed by a standing on ice (or at room temperature) for 5 minutes. The tubes were centrifuged at 12000 rpm and 4° C. for 15 minutes and the supernatant was carefully transferred to a new RNase-free 1.5 ml EP tube (by sucking with a 200 μl pipette without touching the middle layer). Each EP tube was added with isopropanol of an equal volume to the supernatant, and the supernatant and isopropanol were mixed uniformly followed by a standing on ice for 10 minutes. The EP tubes were centrifuged at 12000 rpm and 4° C. for 10 minutes with the supernatant discarded. 1 ml of absolute ethanol was added to mix uniformly with the cells and each tube was centrifuged at 12000 rpm and 4° C. for 5 minutes with the supernatant discarded. Residual liquid in each tube was sucked out with a 10 μl pipette, and dried at room temperature for about 5-10 minutes. Appropriate amount of RNase-free water was added to dissolve RNA according to the quantity of precipitation at the bottom of the tube and the obtained RNA may remain available for long-term storage at −80° C.

RNA validation: an agarose gel (1%) electrophoresis (180V, 10 minutes) was performed, and RNA concentration was detected by Nanodrop and stored for use.

1.1.3.7 Reverse Transcription of RNA into cDNA

The concentration of the extracted RNA was measured (by Nanodrop) to determine the required quantity of RNA for reverse transcription. 2 μg of RNA sample (the amount was generally applied for loading) was added with 1 μl of random primer, 1 μl of dNTPs, and RNase-free water to a volume of 13 μl to form a reaction mixture, which was incubated at 65° C. for 5 minutes. Then, the reaction mixture was added with 4 μl of 5× First buffer and 2 μl of DTT followed by an incubation at 37° C. for 2 minutes to obtain a blend. Next, the blend was added with 1 μl of MLU enzyme on ice and followed by three incubations including at 25° C. for 10 minutes, 37° C. for 50 minutes, and 70° C. for 15 minutes. cDNA obtained after the incubations was stored at −20° C.

### 1.1.3.8 Real-Time Quantitative PCR

20 μl of reaction mixture was prepared, including: 10 μl of qPCR mastermix, 0.5 μl (10 μM) of forward quantitative primer, 0.5 μl (10 μM) of reverse quantitative primer, 1 μl of cDNA, 0.4 μl of ROX and 7.6 μl of ultra-pure water. The real-time quantitative PCR was performed by ABI 7500 Fast.

### 1.1.3.9 Tissue Sectioning and H&E Staining

### 1.1.3.9.1 Paraffin Embedding & Sectioning

The tumor tissue was cut into small pieces of about 5 mm in diameter and placed in an embedding cassette. And then the embedding cassette was soaked in 4% paraformaldehyde for an overnight fixation.

Tissue dehydration: the embedding cassette was removed from formaldehyde, and washed with running water for 30 minutes. The cassette was then soaked in 75% alcohol for 30 minutes, 85% alcohol for 30 minutes, 95% alcohol overnight, and absolute alcohol for an hour and a half, respectively.

Tissue transparenting: the cassette was soaked in dimethylbenzene at room temperature for 35 minutes.

Tissue waxdip: the embedding cassette was soaked in wax tank I at 60° C. for 1-2 hours, and was then transferred to wax tank II for another waxdip of 1 hour.

Tissue embedding: the tissue in the embedding cassette was transferred to a preheated mold (previously added with a small amount of dissolved wax), and the lid of the embedding cassette was placed above the mold. Paraffin was continuously added until the lid of the embedding cassette was completely submerged in the paraffin.

Tissue cooling: the mold was cooled at 4° C., and the embedded tissue in the mold was removed after a complete solidification of the paraffin for a storage at room temperature.

Paraffin sectioning: the tumor tissue was sectioned into sections with a thickness of 5 μm by a paraffin-embedding histotorne. The sections were placed in 45° C. water to unfold and the unfolded sections were picked up with glass slides, with water on the slide gently removed. The slides were dried at 65° C. for 3-4 hours, and were stored at room temperature.

### 1.1.3.9.2 H&E Staining

Section dewaxing: paraffin sections were placed in dimethylbenzene for 1 h.

Section hydration: the sections in dimethylbenzene were transferred and soaked in absolute alcohol for 2 minutes, 95% alcohol for 2 minutes, 85% alcohol for 2 minutes, and 75% alcohol for 2 minutes, respectively. The sections were washed with distilled water for 1 minute.

Section staining: the section staining procedure included hematoxylin staining for 10 minutes, tap water washing, 0.5% eosin staining for 1 min, tap water washing for 2-5 minutes, and distilled water washing for 1-3 s. The staining effect was evaluated by microscopy and the staining process may be repeated to achieve a desirable result.

Section dehydration: the stained sections were sequentially placed in 75% alcohol for 10-30 s, 85% alcohol for 10-30 s, 95% alcohol for 30-60 s, and absolute alcohol for 2-3 minutes to obtain dehydrated section, and the dehydrated sections were examined by microscopy.

Transparent sealing: the dehydrated sections were placed in dimethylbenzene for 15 minutes and removed followed by dripping with gum with dimethylbenzene wetting. Cover slips were applied to the sections (without bubbles) and the sections were dried at room temperature and photographed. The dried sections were suitable for a long-term storage at room temperature.

### 1.1.3.10 Immunofluorescence Staining of Tissue Section

The tissue was embedded and sectioned to obtain sections (referred to 1.1.3.9.1). The sections were dewaxed and hydrated (referred to 1.1.3.9.2). 1-2 drops of hydrogen peroxide (3%) was added onto the sections and the sections were incubated at room temperature for 10 minutes to reduce activity of endogenous peroxidase. And then the sections were washed with PBS 3 times (3 minutes for each washing).

Heat induced antigen repairing: the sections were placed in preheated antigen-repairing buffer, and were microwaved in a strong-heating mode for 5 minutes and then in a defrosting mode for 15 minutes, and the sections were cooled at room temperature.

Blocking: the sections were blocked with 1% BSA at 37° C. for 30 minutes with the serum discarded. The sections were dripped with CD206-FITC (antibody diluted at a ratio of 1:50), and kept at 4° C. in the dark overnight. The sections were washed with PBS 3 times (5 minutes for each washing) and added with DAPI, and then with PBS. Cover slips were applied, and the sections were photographed.

### 1.1.3.11 Multi-Cytokine ELISA Assay

IFN-γ, TNF-α, IL-1β, IL-10, NO, IL-2 and IL-12 in the serum of mice with melanoma were detected by Bio-Plex cytokine detecting system.

1.1.3.12 Macrophage from Tumor Tissue Sorting

Tumor mononuclear cells were isolated to obtain a cell suspension (referred to 1.1.3.3.2) and the cell suspension was added with fluorescence-labeled anti-mouse antibody F4/80-FITC, and was kept in the dark for 30 minutes. The cell suspension was added with 1 ml of 1×PBS and centrifuged at 1500 rpm for 5 minutes. The cells were then resuspended with appropriate amount of sorting buffer according to the amount of cells. Cells labeled with F4/80-FITC were sorted out by using Aris III flow cytometer. The sorted cells were washed with 1×PBS, and were centrifuged at 1500 rpm for 5 minutes with the supernatant discarded. The sorted cells were mixed completely with Trizol and then stored at −80° C. for use.

### 1.1.4 Data Processing & Statistical Analysis

All data in this study were obtained from at least three independent experiments. The experimental data were all presented as means±SD, and were stored in Excel to establish a database. The analysis was performed using statistical software SPSS 13.0. Intra-group comparisons were analyzed using Student's unpaired t-test with probability calculations, and the differences of statistical significance were presented by “p<0.05” (*, P<0.05; **, P<0.01; ***, P<0.001). The statistical charts were all completed by GraphPad Prism Version 5.0 (GraphPad Software Inc, San Diego Calif.). The flow cytometry data was analyzed using FlowJo 7.6.1 software (Tree Star, Inc, USA)

### 1.2 Results

### 1.2.1 Inhibition of FATS Gene Defect on Tumorigenesis and Progression of Melanoma in Subcutaneous B16 Cell in Tumor-Bearing Mice.

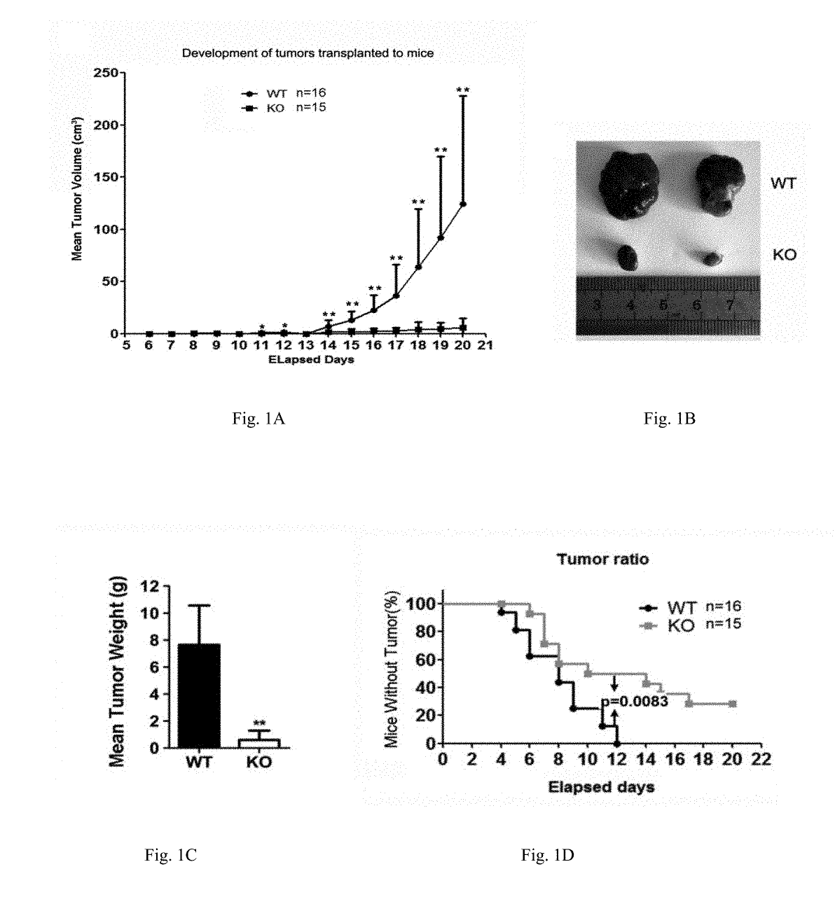

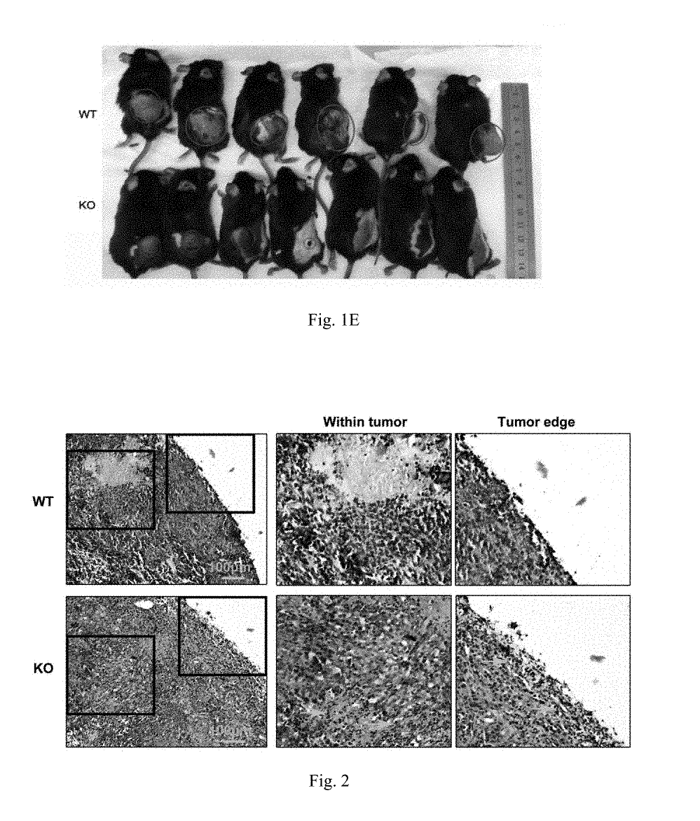

A mouse melanoma model was built to study the role of FATS gene in melanoma. A mouse melanoma xenograft model was built (referring to 1.1.3.2.2 for particular procedures). The results showed that the melanoma formation rate was significantly lower in KO mice than in WT mice (FIGS. 1D-1E, P<0.01). The growth rate, volume and weight of the tumor in the KO mice were also significantly reduced (FIGS. 1A-1B, P<0.01; FIG. 1C, P<0.01). These results indicated that FATS gene defect significantly inhibits the tumorigenesis and progression of melanoma in subcutaneous B16 cell in tumor-bearing mice.

### 1.2.2 Increase of Inflammatory Cell Infiltration in Melanoma Tissue of Mice by FATS Gene Defect

To study the mechanism by which FATS gene inhibits tumorigenesis, H&E staining was first performed on tumor tissue from WT and KO mice to detect inflammatory cell infiltration in the tumor tissues. As shown in FIG. 2, a significant increase of infiltration was observed in inflammatory cell both within and at the edge of the tumor tissue in FATS gene-defect mice indicating that FATS gene defect increases inflammatory cell infiltration in mouse melanoma.

1.2.3 Effect of FATS Gene Defect on Frequency of Immune Cell in Peripheral Immune Organs in Mice with Melanoma

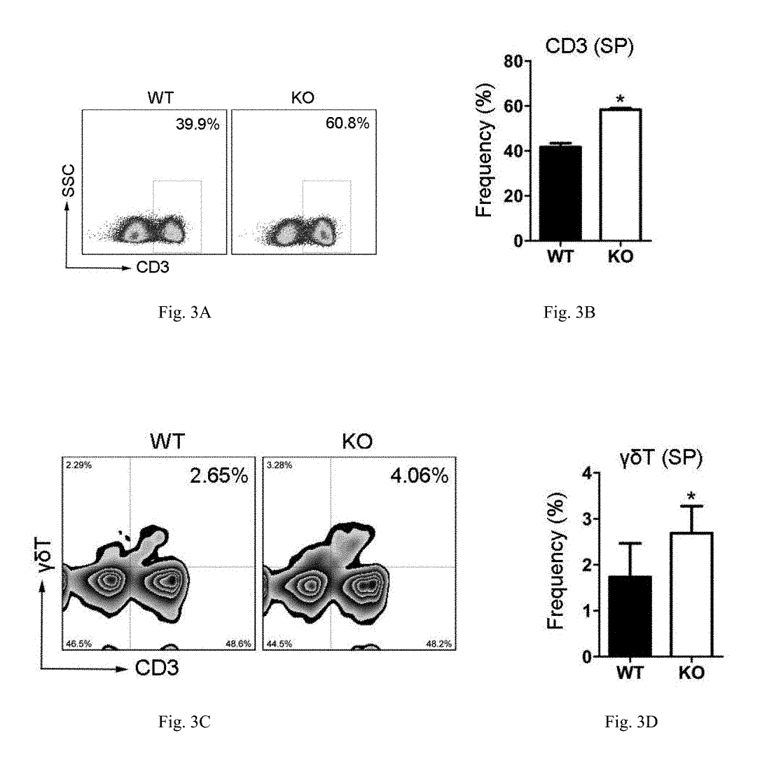

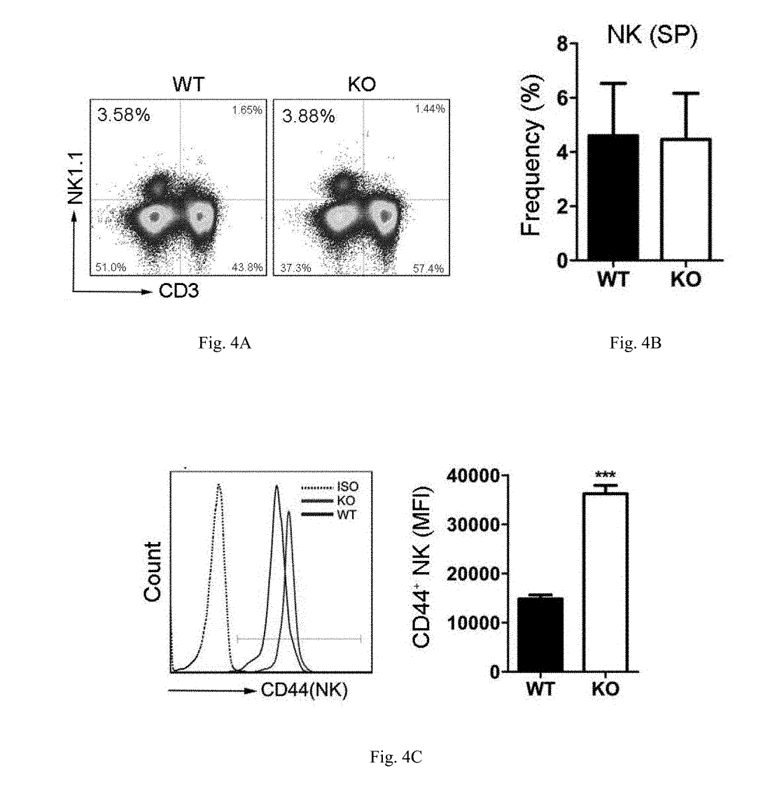

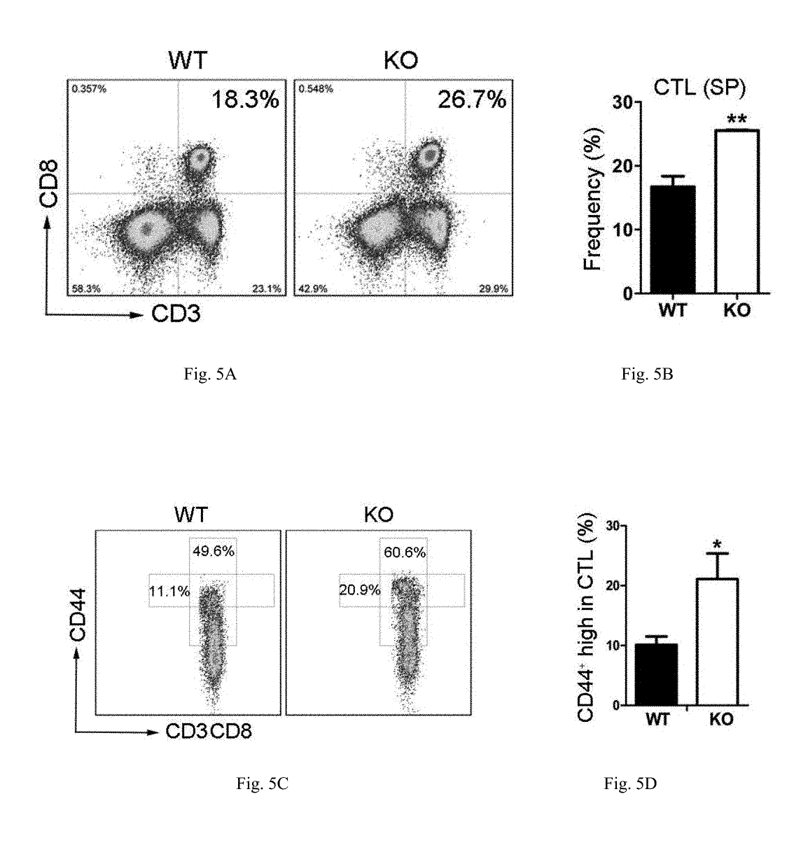

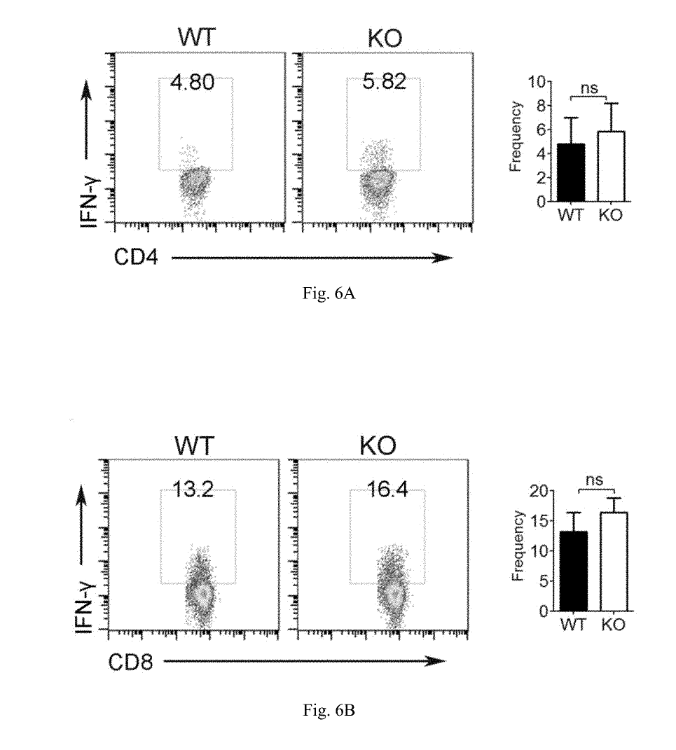

Based on the above results, it is speculated that the inhibition of tumor growth by FATS gene defect may be achieved via effecting tumor-associated immune cells. To prove such speculation, the immune cells in the peripheral and tumor tissues of the WT and KO mice with melanoma were further tested. Spleens (a peripheral immune organ) of tumor-bearing mice were first examined. The mice were sacrificed after 20 days of B16 cell tumor-graft experiment. Mononuclear cells were separated from the spleens and were analyzed by flow cytometry for detecting the changes in various immune cell subsets in the spleens. As shown in FIGS. 3A-3D, the amount of total T cells (CD3⁺ T cells) and γδ T cells (γδ+/CD3⁺) in spleens was significantly increased in KO mice compared to WT mice. Although no significant difference was observed in the amount of natural killer (NK) cells (NK1.1⁺) (FIGS. 4A-4B), the activation level of NK cells (NK1.1⁺/CD44⁺) was significantly increased (FIG. 4C) (positive CD44⁺ indicates activation of T cell and NK cell). In addition, the frequency of CD8⁺ T cells was increased significantly (FIGS. 5A-5B), and the activation level of CD8⁺ T (CD44) was also significantly improved. The horizontal box represents the frequency of CTLs with high expression of CD44, which is higher in the KO mice than in the WT mice (FIGS. 5C-5D). No significant difference was observed in expression in CD8⁺ T cells, or frequency of Th1 cells (CD3⁺CD4⁺IFN-γ⁺) in the spleens of the WT and KO mice (FIGS. 6A-6B). These results indicate that FATS gene defect increases the anti-tumor immunity in peripheral immune organs of mice with melanoma.

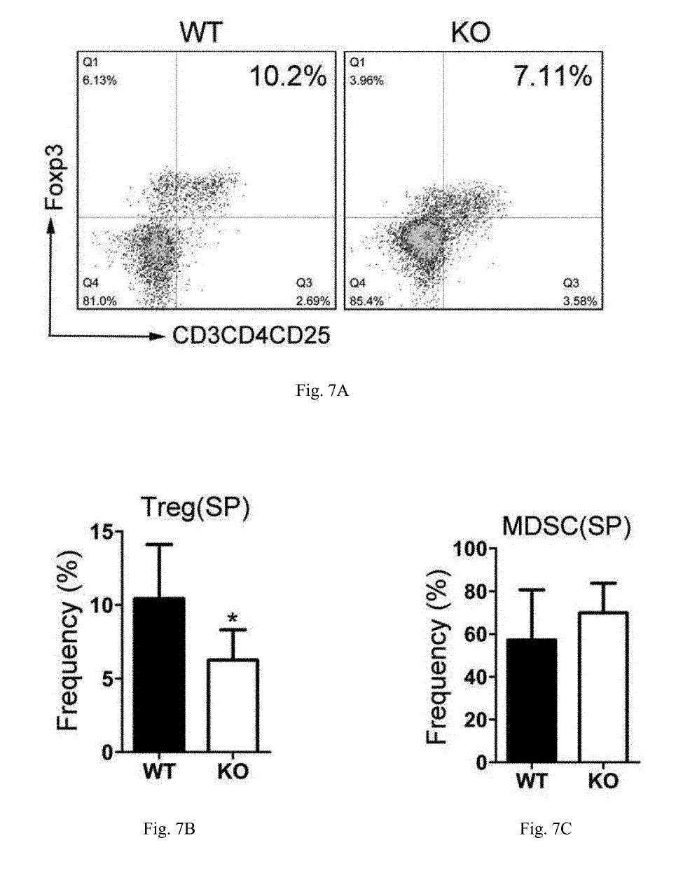

The immune cells in spleens that promote tumor growth, including regulatory T cells (Treg, CD3⁺CD4⁺CD25⁺Foxp⁺) and myeloid-derived suppressor cells (MDSC, CD11b⁺ Ly6C⁺ Ly6G⁺) were tested. Flow cytometry results showed that although frequency of MDSC was not significantly different in the WT and KO mice (FIG. 7C), frequency of Treg was reduced significantly due to FATS gene defect (FIGS. 7A-7B), indicating that FATS gene defect inhibit suppressive tumor immunity.

1.2.4 Increase of Level of Cytokines Associated with Promoting Tumor Killing in Serum of Mice with Melanoma by FATS Gene Defect

Studies have shown that IL-2 can effectively stimulate the proliferation of effector T cells and NK cells, and thus is an important cytokine for T cell proliferation, as well as an important growth factor participating in the proliferation of antigen-activated lymphocytes and the generation of immune memory. IL-12 can promote T cell proliferation and NK cell activation, induce Th1 cell polarization and CTL generation, as well as inhibit angiogenesis. IL-12 can be produced by M1 type macrophages, which can also secrete IL-1β and TNF-α to mediate the lysis of tumor cells. Other Studies have also reported that increase of IFN-γ promotes polarization into M1 type macrophages, inhibits angiogenesis and promotes anti-tumor immune surveillance. The immunosuppressive cytokine (e.g., IL-10) can be produced from immunosuppressive cells (M2 type macrophages, Tregs, etc.), and promotes tumor growth. Based on many studies in the art, a multi-cytokine ELISA assay on the serum from the subcutaneous B16 cell in tumor-bearing WT and KO mice was carried out so as to further study the effect of FATS gene in mouse melanoma. As shown in FIG. 8, IL-2 and IL-12 level was significantly increased in the serum of KO mice with melanoma, which was consistent with an increase in frequency of T cell in the KO mice. In addition, levels of IL-1β, TNF-α, and IFN-γ were also significantly increased, and however the level of immunosuppressive factor IL-10 involved in tumor growth was significantly reduced in the serum of the KO mice. These results indicate that FATS gene defect may affect the frequency and function of immune cells in mice with melanoma, thereby inhibiting the progression of melanoma which was consistent with previous experimental results.

1.2.5 Effect of FATS Gene Defect on Immune Cells in the Tumor Immune Microenvironment of Mice with Melanoma

The above results indicate that FATS gene may play an important role in immune regulation. It is known that in addition to peripheral immune organs, tumor immune microenvironment also plays a crucial role in tumorigenesis and progression of tumors. Therefore, in order to thoroughly investigate the effect of FATS gene in mouse melanoma, the changes in immune cells in the tumor immune microenvironment of melanoma in WT and KO mice were tested.

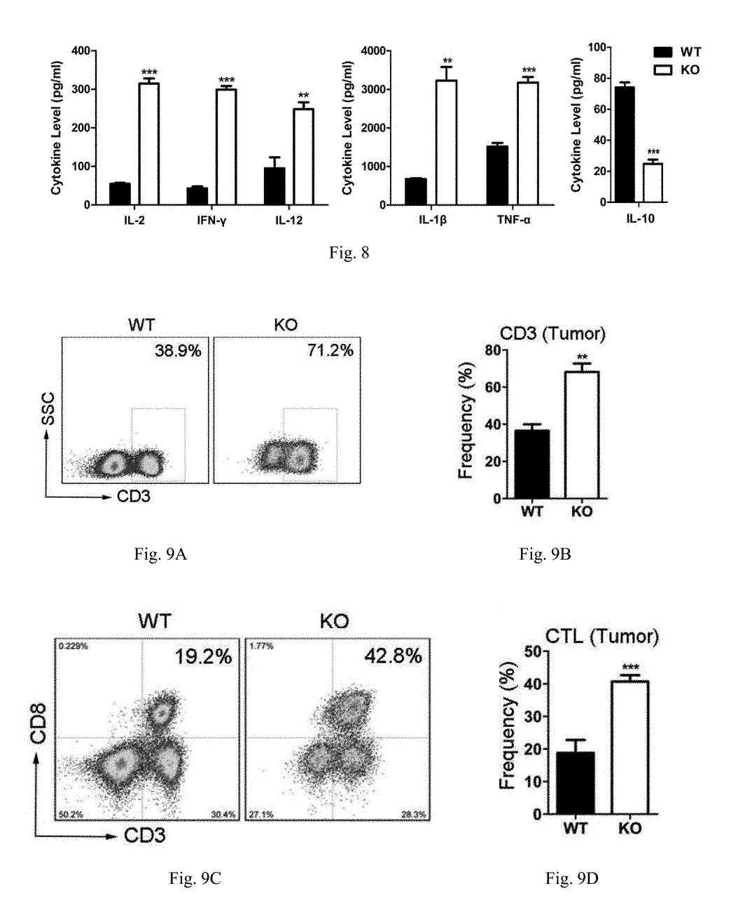

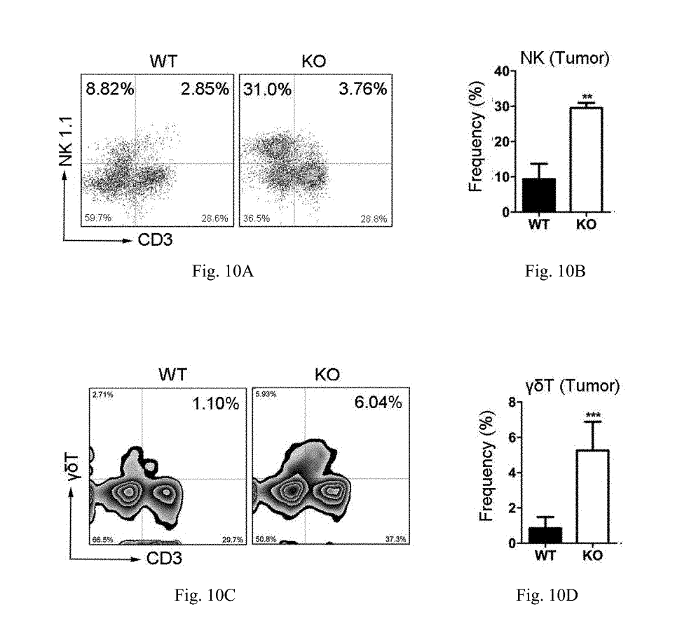

First, the frequency of immune cells with tumor suppressive effect, including CTL, NKT and γδ T cells was analyzed. The results were shown in FIGS. 9-10, the frequency of total T cells (FIGS. 9A-9B) and CTLs with tumor-killing ability (FIGS. 9 C-9D) in the tumor immune microenvironment significantly increased in the KO mice and the frequency of NKT (FIGS. 10A-10B) and γδ T cells (FIGS. 10C-10D) increased significantly, which was more significant than that of the immune cells in spleen. Furthermore, the frequency of NKT cells (FIG. 10, top right) increased in KO mice. It is concluded from these results that FATS gene defect improves the killing ability of tumor immunity in TME of mice with melanoma.

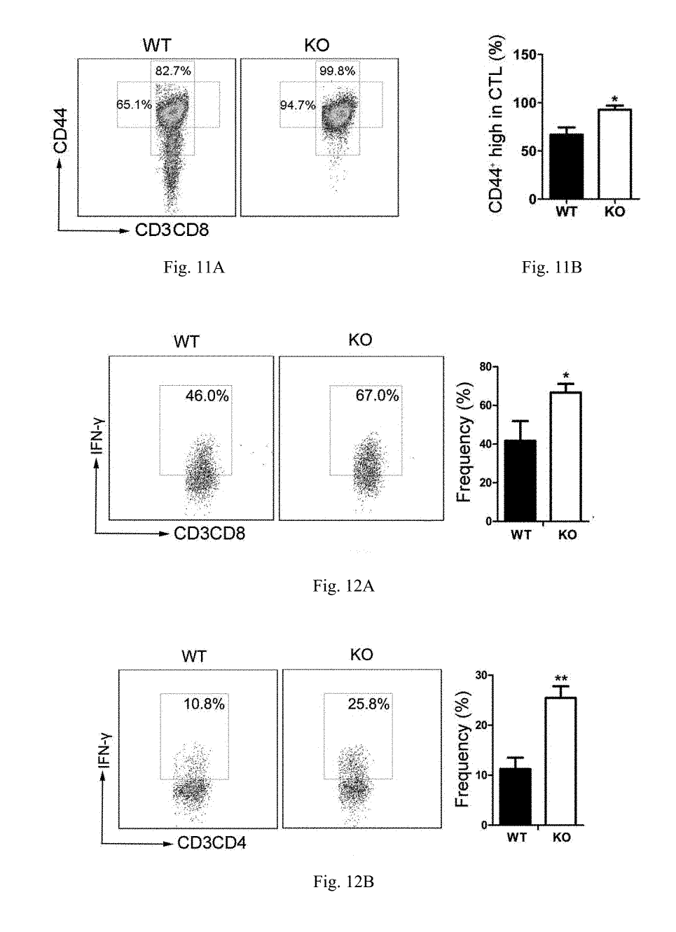

Additionally, the activation level of T cells in tumor was analyzed. The result showed that the activation level of CTLs in the TME of KO mice was significantly improved compared to in WT mice. CD44 substantially exhibit a high expression level in the KO mice, indicating that FATS gene defect improves CTL activation (FIGS. 11A-11B). The result demonstrates that FATS gene defect promotes CTL activation, which was consistent with the tumor suppressive effect in KO mice. Other experimental studies have also indicated that IFN-γ participates in tumor killing and immune surveillance as an important effector of cell killing. The killing effect of CTLs on tumor cells in the tumor microenvironment of WT and KO mice was further analyzed through detection of IFN-γ expression level in CTLs. Th1 cells (CD3⁺ CD4⁺ IFN-γ⁺) was tested. The results showed that IFN-γ expression level was significantly increased in CTLs in the TME of KO mice (FIG. 12A), and the frequency of Th1 cells (CD3⁺ CD4⁺ IFN-γ⁺) in KO mice significantly increased in the TME of KO mice (FIG. 12B), which was significantly higher than increase in the spleens.

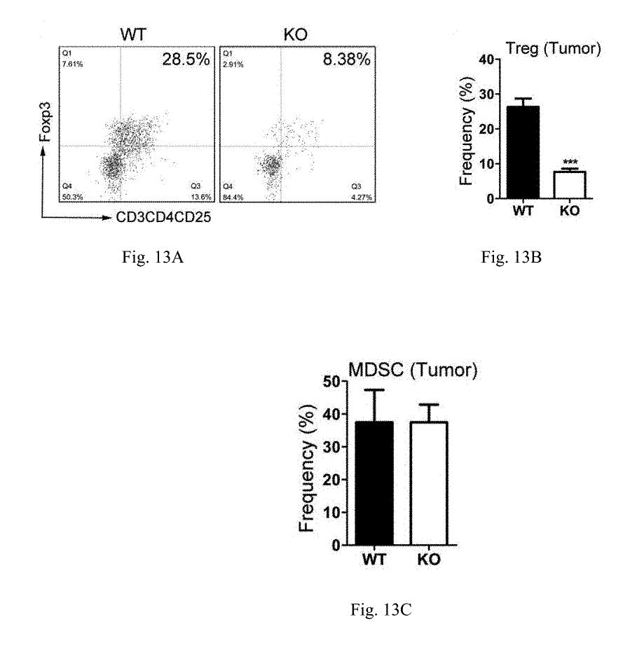

Furthermore, the immune cells that promoted tumor growth in the TME of mice with melanoma, including Tregs and MDSCs were analyzed. As shown in FIG. 13, the frequency of Tregs in the melanoma tumor microenvironment of KO mice was significantly decreased compared to that in WT mice, which was more significant than that in spleens (FIGS. 13 A-13B). There was no significant difference in the frequency of MDSCs (FIG. 13C). This result was consistent with the spleen test, indicating that FATS gene defect significantly inhibit suppressive immunity that promotes tumors.

Tumors contained a large amount of macrophages, called tumor-associated macrophages (TAMs). TAMs were considered to be M2 type macrophages. Studies have shown that M2 type macrophages can promote tumor angiogenesis, invasion and metastasis. Immunosuppression can also be promoted by the production of IL-10. M2 type macrophages express CCL22 and recruit Tregs to inhibit the function of CTLs. M2 type macrophages play an important role in growth, survival and metastasis of tumor cells. In contrast, M1 type macrophages express MHC-II molecules, showing a strong ability of phagocytosis and antigen presentation. In addition, M1 type macrophages produced IL-12, promoted T cell activation and proliferation, inhibited angiogenesis, and cross-presented antigens to CD8⁺ T cells. M1 type macrophages may activate Th1 type responses. Given opposite effect of different macrophage subtypes in tumors, it is necessary to detect the macrophage subtypes in the TME.

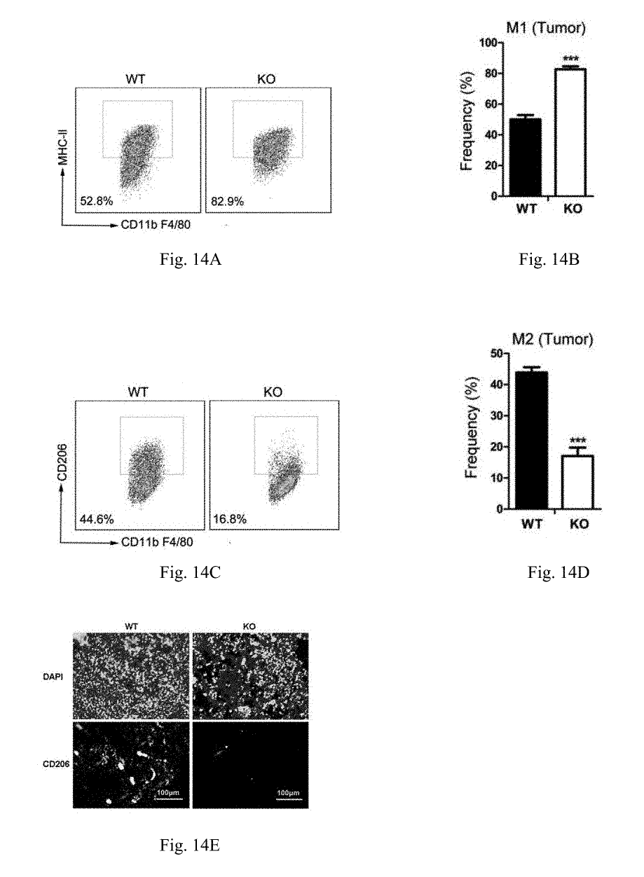

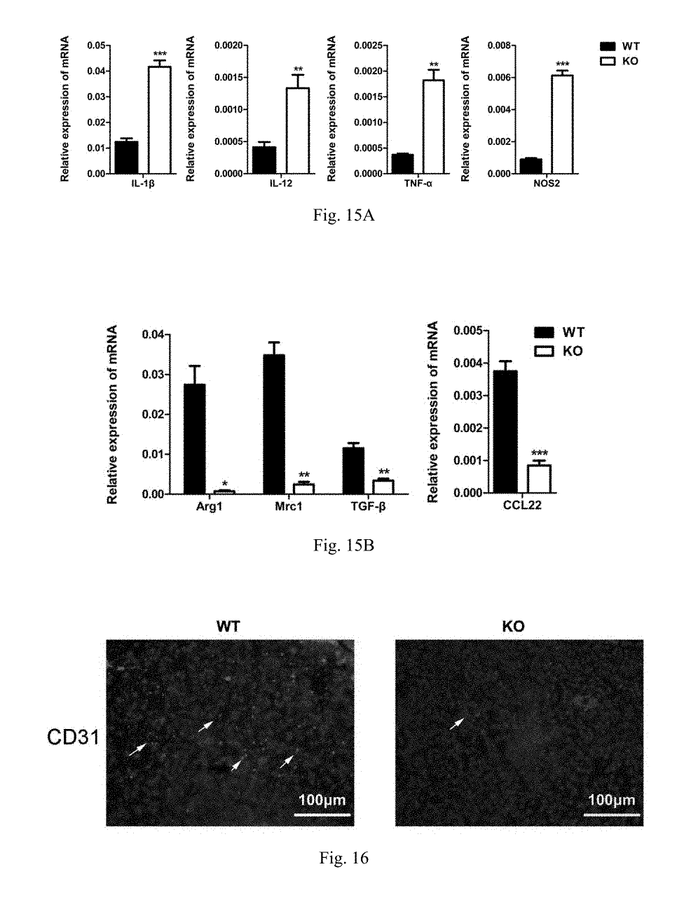

Macrophage typing in tumors (melanoma) of WT and KO mice was analyzed with flow cytometry. As shown in FIG. 14, the result shows that the frequency of M1 type macrophages in the TME was significantly increased in KO mice (FIGS. 14A-14B), and however the frequency of M2 type macrophages was significantly reduced (FIGS. 14C-14D). In addition, immunofluorescence staining on tumor tissue sections with CD206-FITC was conducted (CD206 is an important surface marker of M2 type macrophages). It is found that CD206 level in tumors was significantly higher in WT mice than in KO mice (FIG. 14E). These results indicate that the frequency of tumor suppressive M1 type macrophages was significantly increased in the TME by FATS gene defect, and the frequency of tumorigenic M2 type macrophages was significantly decreased, which was consistent with the inhibition to tumor growth in KO mice.

To further verify the above results, macrophages (F4/80 positive) from tumor tissue were sorted out by flow cytometry. RNAs were extracted. Real-time quantitative PCR was performed to detect the expression level of important genes expressed by M1 type and M2 macrophages, as well as to detect the expression level of cytokines important for M1 type macrophage polarization. As shown in FIG. 15, the expression level of genes expressed by M1 type macrophages (IL-12, TNFα and NOS2) were increased in macrophages of KO mice (FIG. 15A), and however the expression level of genes expressed by M2 type macrophages (IL-10, Agr1, Mrc1 (CD206) and CCL22) was reduced in KO mice (FIG. 15B). This result further demonstrates that mice macrophages with FATS gene defect are more likely to polarize into M1 type macrophages that promote tumor killing, while the polarization into M2 type macrophages that promotes tumor growth was inhibited.

### 1.2.6 Inhibition of FATS Gene Defect on Angiogenesis in Tumor Tissue of Mice

Angiogenesis is effective in the tumor growth. Many studies have reported that M1 type macrophages secrete IL-12 and inhibit angiogenesis, while M2 macrophages promote angiogenesis by secreting VEGF, promoting tumor growth and metastasis. Our experimental results also showed that macrophages mainly present in the tumor microenvironment of KO mice are M1 type macrophages. Therefore, it is speculated that there was a reduction in tumor angiogenesis by FATS gene defect. In order to prove such speculation, the expression level of CD31 in tumor tissue from WT and KO mice was detected through immunofluorescence. As expected, the expression level of CD31 in tumor tissue from KO mice was significantly decreased compared to that in WT mice (FIG. 16). The arrows in FIG. 16 indicate positive CD31, the nucleus revealed by DAPI staining as the background is. These results indicate that FATS gene defect affects tumor angiogenesis by affecting macrophage polarization, and thus ultimately inhibits tumor growth.

### 1.3 Discussion

In recent years, the role of tumor immunity in tumors has been increasingly paid attention to. Anti-tumor immunotherapy can specifically destroy tumor cells, while the functional level of immune system is closely related to tumor treatment and prognosis. Plural studies have indicated the availability of tumor microenvironment as an important target for anti-tumor immunotherapy.

Tumor microenvironment consists of tumor parenchymal cells and interstitial cells, wherein the immune cells in among interstitial cells play an indispensable role in the tumor progression. Tumor growth is affected by interactions between different immune cells, such as antigen presentation and intercellular coordination or inhibition by cytokines. Immune cells coordinate with each other to resist pathogens and tumors. However, tumors create a favorable environment conducive to tumor growth by altering the function of immune cells infiltrating the tumor microenvironment, such evading immune surveillance and resulting in tumor immune escape.

Current studies have shown that immune system plays the dual roles in promoting and inhibiting tumors. As mentioned above, NK cells, neutrophils, γδ T cells, NKT cells, Th1 cells, CTLs and type M1 macrophages are capable of exerting a strong killing effect on tumors.

NK cells and neutrophils are innate immune response cells that directly inhibit tumor growth via perforin, granzyme, Fas/FasL and reactive oxygen species. Meanwhile, γδ T and NK T cells also directly or indirectly exert tumor-killing effect by acting in anti-tumor immunity. In addition, many studies have shown that T cells play a vital role in tumor immunity, wherein naïve T cells can recognize short peptides presented by MHC molecules on the surface of antigen-presenting cells and thereby differentiate into different effector T cells corresponding to the antigens. CD4⁺ T and CD8⁺ T cells can recognize antigenic peptides presented by MHC-II and MHC-I respectively.

After recognizing the presented antigens, naïve CD4⁺ T cells differentiate into different subtypes of effector helper T cells depending on cytokines present in the microenvironment during the activation process. Cytokines secreted during the differentiation of helper T cells can in turn affect the activation of NK cells and CTLs. Helper T cells include Th1, Th2, Th17, etc., which have different roles in tumors. The production of IFN-γ and several other cytokines by Th1 cells can significantly promote cell-mediated immune response, thereby exert a cytotoxic effect and inhibit tumor growth. Increasing evidence has indicated that activation of Th1 cells promotes CTL proliferation, and activation of NK cells, M1 type macrophages and other potential cytotoxic effector cells. In addition, CTL is an important effector cell in anti-tumor immunity, which exhibits direct cell-mediated tumor cytotoxicity after antigen presentation.

The results of our study show a low rate of tumor occurrence (tumorigensis) and tumor growth in B16 cell tumor-bearing KO mice (FIG. 1), suggesting that FATS gene defect affected the tumor-associated immune cells in tumor. Therefore, we comprehensively analyzed the changes of immune cells in peripheral immune organs and tumor immune microenvironment of WT and KO mice. Consistent with the known findings, the frequency of anti-tumor immune cells was significantly increased in KO mice, especially in the tumor microenvironment. This indicates that FATS gene defect was confirmed to substantially affect tumor-associated immune cells. The frequency of tumor-infiltrating inflammatory cells was significantly increased in KO mice (FIG. 2), which was consistent with an increase in the frequency of total T cells in tumor detected by flow cytometry (FIG. 9). Furthermore, the frequency of γδ T and NK cells were also increased upon FATS gene defect (FIG. 10). More importantly, the frequency of major effector T cells (Th1 cells and CTLs) was also significantly increased (FIGS. 9 and 12). Meanwhile, the frequency of cells expressing T cell activation marker CD44 and major effector cytokine IFN-γ was significantly increased among CTLs (FIGS. 11-12). These results suggest that FATS gene defect significantly increased the tumor cytotoxic response, which was consistent with melanoma inhibition in KO mice.

Further experiments found that M1 type macrophages were significantly increased in number in the tumor microenvironment of KO mice (FIG. 14). In recent years, with the deepened studies of macrophages, the role of M1 type macrophages in tumors has been increasingly paid attention to. It is well known that tumor-associated macrophage is one of the major cells consisting tumor microenvironment and poses a crucial influence on tumor growth. Studies have found that type M1 macrophages express MHC-II molecules, and thereby exhibit the functions of phagocytosis and antigen presentation. Meanwhile, M1 type macrophages can produce pro-inflammatory cytokines including iNOS2, ROS, RNS, IL-1β and TNF-α, thereby mediate the cytolysis of tumor cells as playing a role in cell killing. M1 type macrophages produce IL-2 and IL-12; wherein IL-2 can stimulate the proliferation of activated effector T cells and NK cells, and is an important growth factor involved in the proliferation of antigen-activated lymphocytes and generation of immune memory; IL-12 can promote the secretion of IFN-γ by T, NK and NK T cells as IL-12 receptors, and induce Th1 cell polarization and CTL generation, while IFN-γ can further promote the enhancement of the activity of type M1 macrophages by positive feedback regulation. Meanwhile, IFN-γ can also promote anti-tumor surveillance and inhibit oncogene activation and angiogenesis. Other studies have found that IL-12 also exhibits anti-angiogenic activity. Moreover, M1 type macrophages can also cross-present antigens to CD8⁺ T cells. Therefore, the role of M1 type macrophage polarization in anti-tumor immunity becomes extremely important, which effectively combines the innate and adaptive immune responses required for anti-tumor immunity.

Except for immune cells with tumor-killing ability, there are also immune cells promoting tumor growth in the tumor microenvironment. M2 type macrophages, MDCSs, Tregs and other immunosuppressive cells can inhibit anti-tumor immune response, such allowing invasive tumor cells to evade immune surveillance and hinder the tumor immune response.

Macrophages are classified into M1 and M2 types, wherein M2 type macrophages promote tumor angiogenesis, invasion and metastasis. CCL22 expressed by M2 macrophages has a recruiting effect on Tregs, while Tregs further inhibit the function of CTLs. Meanwhile, M2 macrophages can also induce T cells to differentiate into Tregs or other T cell subtypes with no anti-tumor activity by producing TGF-β and IL-10. M2 type macrophage inhibits T cell activation by specifically expressing arginase-1. Arginase-1 (Arg-1) expressed by M2 type macrophages changes arginine into ornithine, which no longer produces NO and thereby reduce the killing ability of macrophages. Therefore, M2 type macrophages play a crucial role in maintaining tumor cell growth, survival and metastasis.

Based on the above theory, we further detected and analyzed the frequency of immunosuppressive cells in tumor immune microenvironment. Consistent with the existing results, our result shows that in the tumor microenvironment of KO mice, although there was no significant difference in MDSC frequency between WT and KO mice, Treg frequency was significantly reduced (FIG. 13). Such result demonstrates that immune response promoting tumor growth was inhibited in KO mice. Interestingly, the detection and analysis on M2 type macrophages found that the frequency of M2 type macrophages was significantly lower in KO mice than in WT mice and immunofluorescence staining also shows consistent result (FIG. 14). The results indicate a difference in macrophage polarization in the tumor microenvironment between WT and KO mice that FATS gene defect may significantly promoted the polarization into anti-tumor M1 type macrophages, and inhibited the polarization into M2 type macrophages.

It is known that in the tumor immune microenvironment, the difference in macrophage polarization (M1/M2) has a profound impact on the tumorigenesis and progression of tumors. Some studies have shown that macrophages in tumors function similarly to macrophages in sterile wounds, that these macrophages do not exhibit killing activity but have a growth-promoting effect. As the researches go deepened, recent studies have suggested that macrophages can polarize into macrophages of different phenotypes depending on immune conditions, that is, microenvironmental stimulation can induce macrophage polarization into M1 or M2 type macrophages. Tumor growth is significantly inhibited when M2 type macrophages in the tumor immune microenvironment are regulated and converted into M1 type macrophages. Studies on human tumors and various mouse tumor models have found that tumor growth can be significantly inhibited via altering the macrophage phenotype from tumor-promoting M2 type to anti-tumor M1 type.

In the present study, the expression level of IL-12, TNF-α, and NOS2 expressed by M1 type macrophages was significantly higher in KO mice than in WT mice, while the expression level of Arg-1, Mrc1 and CCL22 expressed by type M2 macrophages were significantly higher in WT mice than in KO mice (FIG. 15). It is found, though serum ELISA assay for mice, that IL-1β, TNF-α, IL-12 secreted by M1 type macrophages, and level of IFN-γ in KO mice that promotes the activation of M1 type macrophages through positive feedback were significantly increased. Meanwhile, the amount of IL-10 secreted by M2 type macrophages was decreased (FIG. 8). These results suggest that macrophages in the TME of mice tend to polarize into tumor-suppressive M1 type macrophages upon FATS gene defect, indicating the increase and decrease respectively in M1 and M2 type macrophages as a potential cellular mechanism of FATS gene defect for inhibiting melanoma growth. In other words, the inhibition of melanoma by FATS gene defect was probably via direct killing of tumor cells by promoting polarization into M1 type macrophages, and further inhibition and killing of tumor by promoting the proliferation of cytotoxic T cells.

It is suggested that the increase in T cell frequency and the change in M1/M2 macrophage polarization of tumor effect may be a possible cause of the inhibition of melanoma growth in mice upon FATS gene defect. This provides us with a strong basis for further studies on the mechanism of FATS gene's influence on melanoma.

### Example 2 Cellular and Molecular Mechanisms of the Regulatory Effect of FATS Gene Defect on Mouse Melanoma

### 2.1 Subjects and Methods

### 2.1.1 Materials, Reagents and Instruments

### 2.1.1.1 Reagents

| | |
|---|---|
| Apoptosis detection kit: AnnexinV/PI | Sungene Biotech Co., Ltd. |
| assay kit | |
| NO assay kit | Beyotime Co., Ltd. |
| IL-2 ELISA assay kit | Multi Sciences Biotech Co., Ltd. |
| CD3⁺ magnetic beads | Miltenyi Biotech, Germany |
| Radio-immunoprecipitation assay | Sigma, US |
| buffer (RIPA) | |
| Phenylmethanesulfonyl fluoride | Sigma, US |
| (PMSF) | |
| OPI | Sigma, US |
| Lipopolysacchafides (LPS) | Sigma, US |
| Skim milk powder | Tianjin Biomek-Biogo Co., Ltd. |
| Bradford protein quantitative assay | Beijing Biomed Biotech Co., Ltd. |
| kit | |

### 2.1.1.2 Cytokines

| | |
|---|---|
| M-CSF | R&D, US |
| IL-4 | R&D, US |
| IFN-γ | R&D, US |

### 2.1.1.3 Antibodies

| | |
|---|---|
| P65 | Cell Signaling Technology, US |
| IκBα | Cell Signaling Technology, US |
| β-actin | Sungene Biotech Co., Ltd. |
| Histone 3 | Sungene Biotech Co., Ltd. |
| HRP-conjugated goat anti-mouse IgG Ab | Cell Signaling Technology, US |
| HRP-conjugated goat anti-rabbit IgG Ab | Cell Signaling Technology, US |

### 2.1.1.4 Primers

| | Forward primer | Reverse primer |
|---|---|---|
| TNF-α | GAGGCCAAGCCCTGGTATG | CGGGCCGATTGATCTCAGC |
| NOS2 | GTTCTCAGCCCAACAATACAAGA | GTGGACGGGTCGATGTCAC |
| IL-12 | ACAAAGGAGGCGAGGTTCTAA | CCCTTGGGGGTCAGAAGAG |
| Arg1 | CTCCAAGCCAAAGTCCTTAGAG | GGAGCTGTCATTAGGGACATCA |
| CCL22 | CTCTGCCATCACGTTTAGTGAA | GACGGTTATCAAAACAACGCC |
| Mrc1 | CTCTGTTCAGCTATTGGACGC | TGGCACTCCCAAACATAATTTGA |
| Rein1a | CCAATCCAGCTAACTATCCCTCC | ACCCAGTAGCAGTCATCCCA |
| GAPDH | AGGTCGGTGTGAACGGATTTG | GGGGTCGTTGATGGCAACA |

### 2.1.2 Preparation of Reagents

### 2.1.2.1 CFSE Stock Preparation

1 mg of CFSE was dissolved in 1800 μl of DMSO, and the stock was dispensed and stored at −20° C. The stock can be diluted with serum-free medium, 1×PBS or other buffer in use (the specific concentration depends on the experimental requirements).

### 2.1.2.2 Buffer for Magnetic-Beads Cell Sorting (MACS) (0.5% BSA/PBS)

10% BSA was prepared and diluted 20 times with 1×PBS, which was stored at 4° C.

### 2.1.2.3 Working Solutions for Gel Preparation

1) Concentrated gel buffer (1.0 mol/L Tris-HCl, pH 6.8)

12.114 g of Tris (MW 121.14) was dissolved completely in 100 ml of distilled water. Concentrated hydrochloric acid was added to adjust the pH to 6.8 to produce a buffer and the buffer was stored at 4° C. for use.

2) Separation Gel Buffer (1.5 Mol/L Tris-HCl, pH 8.8)

18.671 g of Tris (MW 121.14) was dissolved completely in 100 ml of distilled water. Concentrated hydrochloric acid was added to adjust the pH to 8.8 to produce a buffer and the buffer was stored at 4° C. for use.

3) 10% Sodium Dodecyl Sulfate (SDS)

10 g of SDS was dissolved completely in 100 ml of distilled water. The dissolution can be carried out in a water bath at 50° C. when the SDS was difficult to dissolve, and the solution was stored at room temperature. Precipitation appearing during long-term storage can be dissolved by water-bath heating, which will not affect the use.

### 4) 10% Ammonium Persulfate (AP)

0.1 g of ammonium persulfate was dissolved completely in 1 ml of distilled water and the solution was dispensed in 0.2 ml EP tubes, and was stored at −20° C.

5) 30% Acrylamide

Stored at 4° C.

6) Tetramethylethylenediamine (TEMED) stock solution

Stored at 4° C.

### 2.1.2.4 Buffers for Immunoblot

1) Running buffer (10× concentrated electrophoresis buffer)

144 g of glycine, 30.2 g of Tris-base and 10 g of SDS were fully dissolved in ultra-pure water, and the solution was diluted to 1 L. The solution was stored at room temperature and diluted to 1× for use.

2) Transfer Buffer (1× Transfer Buffer)

14.4 g of glycine and 3.02 g of Tris-base were fully dissolved in ultra-pure water, and the solution was diluted to 800 ml and then added with 200 ml of methanol for use.

3) 10×TBS Buffer (10× Concentrated TBS Buffer)

80 g of NaCl and 24.2 g of Tris-base were fully dissolved in ultra-pure water, and the solution was diluted to 1 L, with the pH adjusted to 7.6 by concentrated hydrochloric acid to obtain a buffer, which was stored at room temperature.

4) 1×TBST Buffer

100 ml of 10× concentrated TBS buffer was added to ultrapure water and diluted to 1 L. The solution was then added with Tween-20 and mixed completely. Tween-20 should be sucked out gently to prevent the formation of air bubbles for its relatively high viscosity and the solution should be continuously stirred with a glass rod to prevent Tween-20 from sinking quickly to the bottom of the beaker during the dissolution.

5) Blocking Buffer/Antibody Diluent (5% Skim Milk)

5 g of skim milk powder was fully dissolved in 100 ml of 1×TBST buffer to obtain a blocking buffer. The buffer can be stored at 4° C. for a week, and can also be available for a long-term storage at −20° C.

2.1.3 Experimental Methods

### 2.1.3.1 Co-Culture of Macrophages and T Cells

### 2.1.3.1.1 Tumor Tissue Macrophages Sorting

Mouse tumor mononuclear cells were separated (referring to 1.1.3.3.2 for specific procedures). Fluorescence-labeled rat anti-mouse antibody F4/80-FITC was added to the cell suspension and then cells labeled with F4/80-FITC were sorted out by Aris III flow cytometer. The cells obtained were cultured in sterile 1640 medium containing 10% serum for use.

### 2.1.3.1.2 Mouse CD3⁺ T Cell Sorting

Spleens of normal wild type C57 mice were separated and used for the separation of mononuclear cells (referring to 1.1.3.3.1 for specific procedures). The cell suspension was added with 3-5 ml of magnetic bead sorting buffer, and was centrifuged at 300 g for 10 minutes. The supernatant was removed completely and the cells were added with magnetic beads sorting buffer (10 times the volume of the magnetic beads added) and CD3⁺ magnetic beads (10 μl/10⁷ cells). The mixture was mixed well and left standing at 4° C. for 15 minutes, during which the mixture was subjected to another mix. The mixture was added with 20 ml of magnetic bead sorting buffer, and was centrifuged at 300 g for 10 minutes. The supernatant was removed completely and the sorting buffer was added (500 μl/10⁸ cells) to the cells, and the mixture was added dropwise to a sorting column pre-rinsed by the sorting buffer. The sorting column was placed above a sterile 15 ml centrifuge tube, and was added with 2 ml of 1640 medium containing 10% FBS to quickly wash out the cells adhering to the column, and then the cells were counted. The cells obtained were CD3⁺ T cells, and were cultured for use.

### 2.1.3.1.3 CFSE Staining

The sorted CD3⁺ T cells were resuspended to a cell concentration of 1×10⁶/ml. CFSE stock solution was added (2 μl per ml of cells), and the mixture was mixed uniformly for a final concentration of 10 μM and incubated at 37° C. for 10 minutes. The mixture was removed from incubation, and was added with a pre-cooled medium of a 5 times volume to stop the staining. The mixture was incubated on ice for 5 minutes, and was centrifuged at 1300 rpm for 5 minutes. The supernatant was discarded, the cells were added with 1640 medium containing 10% FBS and were washed therewith 3 times, and each time for centrifuging at 1300 rpm for 5 minutes and discarding the supernatant. The cells were resuspended and the cell concentration was adjusted according to the experimental requirements.

### 2.1.3.1.4 Co-Culturing Macrophages and T Cells

Macrophages sorted by flow cytometry were treated with mitomycin C (5 μg/μl), the cells were added with 12.5 μl/1×10⁶ cells mitomycin, and were cultured in a cell culture incubator for 30 minutes. 1640 medium containing 10% FBS was added to wash the cells 3 times, and the cells were counted. The macrophages were co-cultured with CD3⁺ T cells for 3 days, and the ratio of macrophages to T cells was 1:4, and 50-100 μl of medium was added during the 2nd to 3rd day of the culture. The cells were collected after the culture, and were analyzed by flow cytometer along with the primary T cells fixed without passage, for detecting the status of CD3⁺ T cells stained by CF SE.

### 2.1.3.2 Co-Culture of Macrophages and B16 Cells

### 2.1.3.2.1 Tumor Tissue Macrophage Sorting (Referring to 2.1.3.2.1)

### 2.1.3.2.2 Co-Culturing Macrophages and B16 Cells

Macrophages sorted by flow cytometry were treated with mitomycin C (5 μg/μl), and the cells were added with 12.5 μl/1×10⁶ cells mitomycin, and were cultured in a cell culture incubator for 30 minutes. 1640 medium containing 10% FBS was added to wash the cells 3 times and the cells were counted. The macrophages were co-cultured with B16 cells in a ratio of 40:1 for 3 days. The cells were collected after the culture, and were analyzed by flow cytometer for detecting the apoptosis of B16 cells.

### 2.1.3.3 T Cells Proliferation Experiment

A 96-well plates was coated with anti-CD3 and anti-CD28 antibodies (anti-CD3 antibody: 5 μg/ml; anti-CD28 antibody: 2 μg/ml) at 4° C. overnight. CD3⁺ T cells were sorted (referring to 2.1.3.2 for specific procedures). 2), counted, and stained by CFSE. The cells were cultured in the coated 96-well plate (10⁵ T cells per well) for 3 days, and were analyzed by flow cytometer for detecting the status of T cells stained by CFSE.

### 2.1.3.4 Bone Marrow Cells Separation

The mice were sacrificed by cervical vertebra luxation after anesthesia, bilateral lower limbs were disinfected with 75% alcohol, and the femur and tibia were separated and placed quickly in cold PBS. The residual tissue on the bone surface was removed, and the bones were placed in a clean bench. The medullary cavity was exposed by cutting open the ends of the femur along the joint. The bone marrow was flushed into a culture dish with 1×PBS by a 1 ml syringe, and was washed repeatedly therewith until the tibia turned white. The bone marrow was blown to single cells with a 1 ml pipette, and the cell suspension was collected in a sterile 15 ml centrifuge tube and centrifuged at 1000 rpm for 5 minutes. The supernatant was discarded and the cells were added with 1×PBS and centrifuged at 1000 rpm for 5 minutes. The supernatant was also discarded and the cells were resuspended with 1640 medium containing 10% FBS, and were cultured in a cell incubator.

### 2.1.3.5 Directed Differentiation of Macrophages

The separated bone marrow cells added with M-CSF (10 ng/ml), and were cultured for 5 days (half of the medium was changed on the 3rd day of culture), and at this time the cells were M0 type macrophages.

Polarization into M1 type macrophages: M0 type macrophages were added with IFN-γ (20 ng/ml) and cultured for 12 hours, and then were added with LPS (100 ng/ml) and cultured for 4 hours to polarize into M1 type macrophages.

Polarization into M2 type macrophages: M0 type macrophages were added with IL-4 (20 ng/ml) and were cultured for 16 hours to polarize into M2 type macrophages.

### 2.1.3.6 Detection of Macrophage Types

The cells to be tested were collected, and were centrifuged at 1500 rpm for 5 minutes. The supernatant was discarded and the cells were resuspended with 1 ml of 1×PBS, and were centrifuged at 1500 rpm for 5 minutes. The supernatant was discarded and the cells were dispensed to a blank tube, a single-staining tube and an isotype test tube. Each tube was added with 1×PBS till a volume of about 100 μl. A surface staining was performed according to: \textcircled{1} M1 type macrophages: CD11b-FITC/MHC-II-PE/F4/80-APC; CD11b-FITC/CD11c-PE/F4/80-APC, and \textcircled{2} M2 type macrophages: CD206-FITC/CD11b-PE/F4/80-APC (referring to 1.1.3.4 for specific procedures). The cells were analyzed by a flow cytometer.

### 2.1.3.7 Cell Apoptosis Detection

The cells to be tested were collected and washed once with cold 1×PBS (1500 rpm centrifugation for 5 minutes). The supernatant was discarded and the cells were resuspended in diluted binding buffer, and were centrifuged at 1500 rpm for 5 minutes. The cells were dispensed in a blank tube and two single-staining tubes. Each tube was added with 5 μl of AnnexinV-FITC/APC, and was kept at room temperature in the dark for 10 minutes. Then each tube was added with 5 μl of PI-PE, and was kept at room temperature in the dark for 10 minutes. Finally, each tube was added with 200 μl of binding buffer, and was analyzed by a flow cytometer (within 1 h).

### 2.1.3.8 Cellular RNA Extraction (Referring to 1.1.3.7)

2.1.3.9 Reverse Transcription of RNA into cDNA (Referring to 1.1.3.8)

### 2.1.3.10 Real-Time Quantitative PCR (Referring to 1.1.3.9)

### 2.1.3.11 Cellular Protein Extraction

Cells were collected and centrifuged at 1200˜1300 rpm for 5 minutes, and the supernatant was discarded. The cells were blown with 1 ml of 1×PBS and then the cell suspension was transferred to a 1.5 ml EP tube for a centrifugation at 1300-1500 rpm for 5 minutes. The supernatant was removed as cleanly as possible and the cells were blown in RIPA lysing buffer supplemented with PMAF and OPE, and the volume of the RIPA lysing buffer was adjusted according to the amount of the cells. The mixture was left standing for 30 minutes on ice, and was centrifuged at 14,000 rpm and 4° C. for 15 minutes. The supernatant was collected and dispensed in 0.2 ml EP tubes.

### 2.1.3.12 Protein Concentration Detection

1) Protein was used in 10-time dilution according to the table below:

| | Standard protein (μl) | | | | | | | |
|---|---|---|---|---|---|---|---|---|
| | 0 | 1 | 2 | 4 | 8 | 12 | 16 | 20 |
| Ultra-pure water (μl) | 20 | 19 | 18 | 16 | 12 | 8 | 4 | 0 |

2) Preparation of Working Buffer:

Buffer A and B were mixed upside down in a ratio of 50:1 to form a working buffer. Each well in a 96-well plate was added with 200 μl of working buffer and each well was then added with standard protein and diluted protein. The plate was placed at 37° C. for 30 minutes, and was detected for OD value (595 nm) by a microplate reader.

### 2.1.3.13 Immunoblot

1) SDS-PAGE gels preparation: 12% separation gel and 5% concentrated gel were prepared.

2) Electrophoresis: 25-30 μg of protein was added with concentrated loading buffer and ultra-pure water (diluting the buffer to 1×). The mixture was mixed well, and was denatured at 99° C. for 5 minutes. The mixture was then added into a immunoblot gel readily mounted on an electrophoresis device with running buffer. The electrophoresis device was turned on with a running voltage of 80 V (the running voltage was set to 100 V when the protein ran into the separation gel) and the electrophoresis was stopped when the leading edge of the bromophenol blue dye ran to the lower edge of the separation gel.

3) Film transfer: the gel was gently transferred and soaked in transfer buffer. A PVDF film larger than the gel block was cut and activated in anhydrous methanol for 30 s, and was soaked in the transfer buffer for use. The following materials or components pre-soaked in the transfer buffer were placed in a transfer clip in the following order: sponge, filter paper, gel, PVDF film, filter paper and sponge, and the transfer clip was clamped tight to remove any air bubble between each layer. The transfer clip was put into the transfer tank with the film towards the positive electrode and glue towards the negative pole. The power was turned on, with the voltage of 89 V for 60 minutes.

4) Blocking: the PVDF film was immersed in blocking buffer and shaken at room temperature for 1 h.

5) Film washing and hybridization:

Adding primary antibody: the primary antibody was diluted in the antibody diluent according to the instructions of monoclonal antibody. The PVDF film with protein was incubated in the primary antibody, and was shaken overnight at 4° C. and then the film was washed by TBST for 5-10 minutes 3 times.

Adding secondary antibody: horseradish peroxidase (HRP)-labeled goat anti-rabbit antibody or murine IgG antibody (1:2000 dilution) was prepared. The washed PVDF film was incubated in the secondary antibody, and was shaken at room temperature for 2 hours. The film was washed with TBST for 10 minutes 3 times.

6) Exposure: the two reagents from the chemiluminescence kit were mixed in a ratio of 1:1 to form a reaction mixture, and the reaction mixture was then dropped onto a piece of plastic wrap. Filter paper was used to remove (by absorbing) the TBST on the PVDF film and the film was placed with the protein-face facing down onto the reaction mixture for 30 s. The film was incubated with a nitrocellulose film in a dark room for 1 minute and the reaction solution on the film was removed with filter paper. The film was wrapped with plastic wrap with the protein face facing up, and was placed in a compression cassette. The photographic film was exposed in the tablet in a dark room, and the exposure time was determined according specific experiments. The exposed photographic film was developed in a developing solution, and was then fixed in a fixing solution and rinsed with water and dried.

### 2.1.3.14 ELISA Assay

The supernatant of the co-cultured cells was collected, and was centrifuged at 1500 rpm, and the supernatant was collected. 6 standards of 100 μl each were sequentially added to the coated microplate with labels marked and then processed samples were sequentially added in the microplate (100 μl per well).

The microplate was covered by a film, and was incubated at 37° C. for 60 minutes. The liquid in the wells were discarded with the residual completely absorbed by tissue paper. The microplate was washed 5 times with the cleaning solution diluted according to the instructions beforehand. Each well was added with 50 μl substrate I, and was then added with 50 μl substrate II and was mixed well, and the microplate was kept in the dark at room temperature for 15 minutes. 50 μl of stopping solution was added to each well, and was mixed well to terminate the reaction and the OD value (450 nm) was measured by a microplate reader.

### 2.1.3.15 NO Detection

The standard sample was diluted to 1 mM with medium (1 M in stock), and nine 0.2 ml EP tubes were applied (with content shown in the table below). The standards and samples were added to a 96-well plate (50 μl per well) and each well was added with 50 μl of solution I and 50 μl of solution II. The plate was kept in the dark at room temperature for 30 minutes. OD value (540 nm) was detected by a microplate reader.

| | Standard protein (μl) | | | | | | | | |
|---|---|---|---|---|---|---|---|---|---|
| | 0 | 0.1 | 0.2 | 0.5 | 1 | 2 | 4 | 6 | 10 |
| Ultra-pure water (μl) | 100 | 99.9 | 99.8 | 99.5 | 99 | 98 | 96 | 94 | 90 |

### 2.1.4 Data Processing & Statistical Analysis

All data in this study were obtained from at least three independent experiments. The experimental data were all presented as means±SD, and were stored in Excel to establish a database. The analysis was performed using statistical software spss13.0. Intra-group comparisons were analyzed using Student's unpaired t-test with probability calculations, and the differences of statistical significance were presented by “p<0.05” (*, P<0.05; **, P<0.01; ***, P<0.001). The statistical charts were all completed by GraphPad Prism Version 5.0 (GraphPad Software Inc, San Diego Calif.). The flow cytometry data was analyzed using FlowJo 7.6.1 software (Tree Star, Inc, USA).

### 2.2 Results

### 2.2.1 Promotion of Macrophages in FATS Gene Defect Mouse Melanoma on T Cell Proliferation

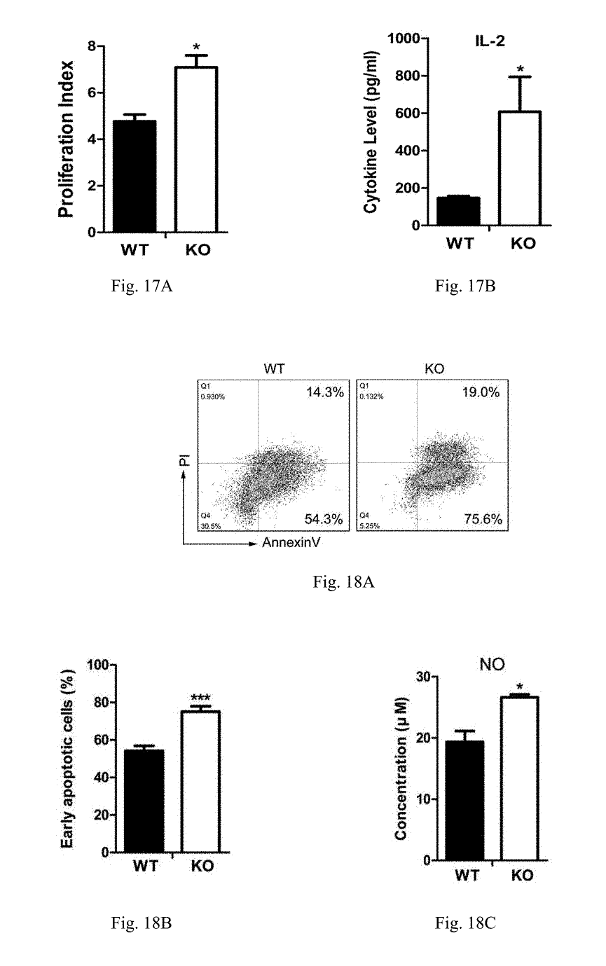

We found that upon FATS gene defect, the frequency of M1 type macrophages in TME increased significantly, while the frequency of M2 type macrophages decreased significantly. Studies have shown that M1 type macrophages can produce IL-2, IL-12 and other cytokines, and IL-2 can stimulate the proliferation of activated effector T cells. Therefore, we speculated that FATS gene defect could regulate the number and frequency of T cells by regulating macrophage polarity. We examined the antigen-presenting ability of macrophages in the TME in order to investigate whether FATS gene defect macrophages could induce the increase in T cells in TME. We co-cultured macrophages sorted from tumor tissue of WT and KO mice with CF SE-labeled CD3⁺ T cells from WT mice and the proliferation status of the T cells was detected by flow cytometry 3 days later. The result shows that macrophages in the TME of KO mice promoted T cell proliferation more significantly than macrophages in the TME of wild-type mice (FIG. 17A). Meanwhile, IL-2 content was found to be higher in the supernatant of FATS gene defect macrophages co-culture than in that of WT macrophages co-culture (FIG. 17B), such confirming the increased T cell proliferation in the co-culture with FATS gene deficient macrophages. These results indicate that macrophages in the TME of FATS gene defect mice exhibited stronger antigen-presenting ability than those of wild-type mice.

### 2.2.2 Stronger Direct Killing Effect of Macrophages in Tumor of FATS Gene Defect Mice on B16 Cells

It is known that in addition to antigen presentation, M1 type macrophages also promoted T cell proliferation and played an important role in innate immunity, and they can produce NO and thereby exhibit a direct killing effect on cells. M2 type macrophages cannot produce NO and thus no longer induced a killing effect on cells. Based on the experimental results obtained, we further examined whether macrophages exhibited the cell killing ability as M1 type macrophages upon FATS gene defect. We sorted out macrophages from melanoma tissue of WT and KO mice by flow cytometry and co-cultured them with B16 cells and apoptotic of B16 cells were detected 1 day later. As shown in FIG. 18, the result shows increased apoptosis of B16 cells co-cultured with FATS gene defect macrophages (FIGS. 18A-18B). Meanwhile, we detected NO level in the co-culture supernatant with consistent result, that NO content in the co-culture supernatant of FATS gene defect macrophages was significantly higher (FIG. 18 C). Such result further indicates that FATS gene defect macrophages had a more potent cell killing ability, that is, the FATS gene defect macrophages tended to polarize into M1 type macrophages more capable of killing cells. In summary, these results were consistent with those of our in vivo experiments.

2.2.3 Promotion and Inhibition of FATS Gene Defect on the Differentiation of Bone Marrow Cells into M1 Type Macrophages and the Differentiation into M2 Type Macrophages Respectively

We found that the frequency of M1 type macrophages in tumor significantly increased upon FATS gene defect, while the frequency of M2 type macrophages was significantly reduced. Therefore, we speculated that FATS gene defect may directly affect macrophage polarization. To verify such hypothesis, we separated the bone marrow cells from WT and KO mice and added M-CSF for directed differentiation into macrophages. On the 5th day of differentiation, the cells were added with IFN-γ and LPS, or IL-4 to further polarize into M1 or M2 type macrophages respectively. The cells were collected 16 hours later, and were comprehensively analyzed for the effect of FATS gene defect on the polarization into M1 and M2 type macrophages.

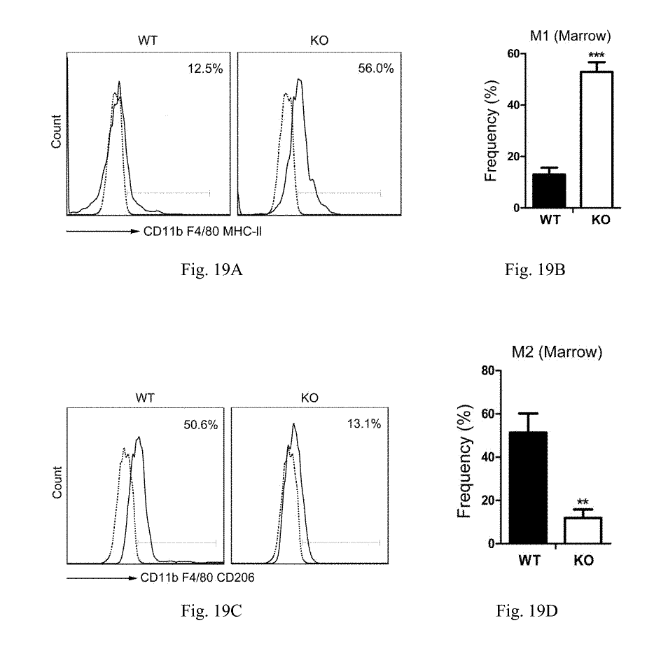

First, we detected and analyzed the frequency of M1 and M2 type macrophages in WT and KO mice by using flow cytometry. As shown in FIG. 19, the result shows that under M1 type polarization condition, the frequency of M1 type macrophages in KO mice was much higher than that in WT mice (FIGS. 19A-19B). Such result indicates that macrophages apparently tended to polarize into M1 type macrophages upon FATS gene defect. Furthermore, the result also shows that under M2 type polarization conditions, the frequency of M2 type macrophages was significantly lower in KO mice than in WT mice (FIGS. 19C-19D). Such result confirmed that FATS gene defect can directly regulate the macrophage polarization, and specifically it promoted the polarization into M1 type macrophages and inhibited the polarization into M2 type macrophages.

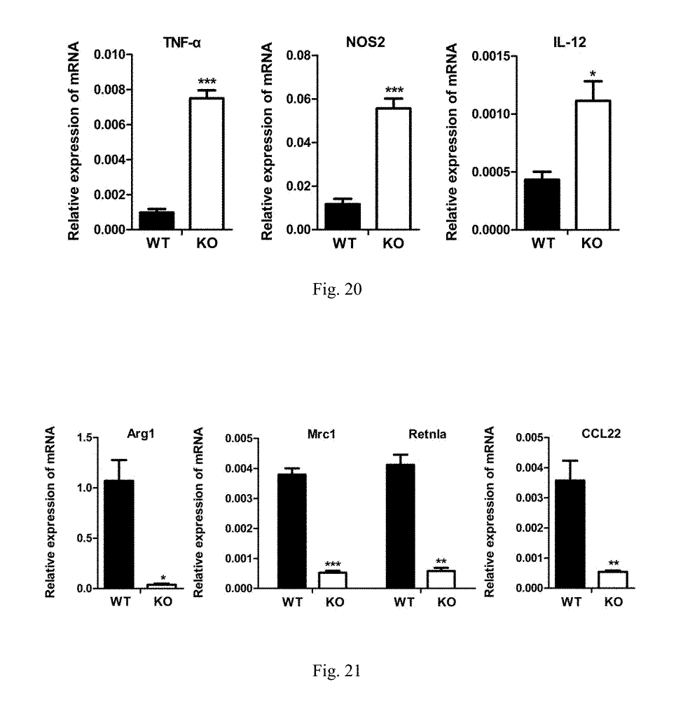

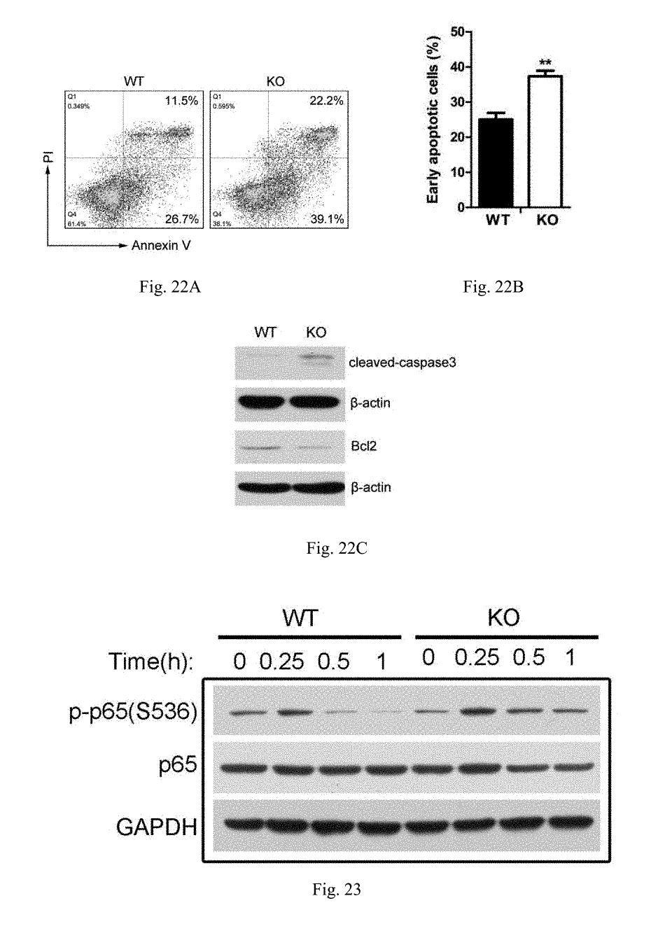

Meanwhile, we also detected the expression level of M1 and M2 type macrophage-associated genes in WT and KO mice under different polarization conditions by RT-PCR. As shown in FIGS. 20-21, the result was consistent with that of the flow cytometry, that the mRNA level of IL-12, TNF-α and NOS2 in M1 type macrophages was significantly higher in KO mice than in WT mice upon the polarization of M0 type macrophages into M1 type (FIG. 20); while upon the polarization into M2 type macrophages, the mRNA level of cytokines expressed by M2 type macrophages (Arg1, Mrc1, Retnla and CCL22) was significantly lower in KO mice than in WT mice (FIG. 21). Consistent with the results of our previous in vivo experiments, these results indicate that FATS gene defect significantly promoted macrophage polarization into M1 type macrophages, and at the same time inhibited the polarization into M2 type macrophages.

### 2.2.4 Promotion of FATS Gene Defect on the Apoptosis of Type M2 Macrophages

We found from the above experiments that M2 type macrophages in KO mice were significantly reduced. In order to further investigate the effect of FATS gene on M2 type macrophages and the underlying mechanisms thereof, we continued to detect and analyze cell apoptosis during macrophage polarization into M2 type macrophages in WT and KO mice. We separated mouse bone marrow cells and added M-CSF to induce directed differentiation into macrophages and the cells were then added with IL-4 to polarize into M2 type macrophages, which were collected and analyzed by flow cytometry for cell apoptosis level. An immunoblot assay was also performed on the collected M2 type macrophages for the expression level of apoptosis-related signals. The result of flow cytometry shows a significant increase in cell apoptosis (in terms of both early and late apoptosis) during the polarization into M2 type macrophages upon FATS gene defect (FIGS. 22A-22B). Such result indicates that FATS gene defect promoted the apoptosis of M2 type macrophages, which was a possible cause of the decrease in the frequency of M2 type macrophages in KO mice. Moreover, we examined the expression of apoptosis-related signals in type M2 macrophages from WT and KO mice. Consistent with the flow cytometry result, and the expression level of Cleaved-caspase3 protein was increased in M2 type macrophages from KO mice compared to that from WT mice. Meanwhile, the expression of apoptosis-suppressive Bcl2 protein was inhibited in the FATS gene deficient macrophages (FIG. 22 C). It is suggested that FATS gene defect promoted apoptosis in M2 type macrophages and thereby reduced the frequency of M2 type macrophages, which was consistent with the decrease in the frequency of M2 type macrophages detected in vitro and in TME detected in vivo.

### 2.2.5 Promotion of FATS Gene Defect on the Activation of NF-κB Signaling Pathway in Macrophages

To further investigate the mechanism by which FATS regulates macrophage polarization, we examined signaling pathways associated with macrophage polarization. The activity of NF-κB signaling pathway was examined during the polarization of bone marrow-derived macrophages into M1 type macrophages. As shown in FIG. 23, p-p65 was significantly increased in the NF-κB signaling pathway upon FATS gene defect (FIG. 23). Such result indicates that FATS gene defect activated the NF-κB signaling pathway in macrophages and promoted the polarization of M0 type macrophages into M1 type macrophages, and thereby increased the frequency of M1 type macrophages.

### 2.3 Discussion

In an in vivo experiment, we analyzed the immune cells in the tumor microenvironment of mice with melanoma, and found that FATS gene defect inhibited immunosuppressive cells that promote tumor growth, and promoted immune cells having a killing effect on tumors. It is well known that immune cells in the TME exert synergistic or inhibitory effect on tumors through antigen presentation and secretion of various cytokines, and macrophages (M1 type macrophages) and effector T cells (CTL, Th1) play important roles in tumor killing and inhibiting tumor growth. We noticed that upon FATS gene defect, the frequency of Th1 and CTLs with major cell killing effect, and M1 type macrophages with both intrinsic killing and antigen presenting functions significantly increased in mice with melanoma. Studies have also shown that M1 type macrophages can produce IL-2, IL-12 and other cytokines, and IL-2 can stimulate the proliferation of activated effector T cells. Therefore we speculated that FATS gene defect may regulate the number and frequency of T cells by regulating macrophage polarity. To verify such hypothesis, we separated macrophages from tumor (melanoma) of WT tumor-bearing and KO tumor-bearing mice; the separated macrophages were co-cultured with CD3⁺ T cells from normal WT mice. We found that FATS gene deficient macrophages significantly promoted the proliferation of T cells, while cytokine IL-2 content in the culture supernatant also increased (FIG. 17). Given such result, we speculated that FATS gene further regulated the proliferation of T cells by regulating macrophage polarity, and thereby affected the frequency of immune cells in the tumor immune microenvironment.

Subsequently, we found from our in vitro experiments that compared to WT bone marrow cells, FATS gene deficient bone marrow cells were more likely to polarize into M1 type macrophages and inhibit polarization into M2 type macrophages under the same polarization conditions during the directed differentiation of bone marrow cells (FIG. 19). Meanwhile, consistent result was also obtained by genetic assay, that: under M1 type polarization conditions, FATS gene deficient macrophages showed a higher level of type M1-specific markers (FIG. 20), including the secreted IL-12 which is crucial for the proliferation and survival of T cells. While under M2 type polarization conditions, the expression of factors Arg-1, CCL22, Mrc1 and Retnla specific for M2 type macrophages was significantly inhibited in FATS gene deficient macrophages (FIG. 21), wherein CCL22 recruits Treg to inhibit tumor effector cells and thereby promotes tumor growth. These results are completely consistent with the data obtained from our in vivo experiments analyzing the changes in the frequency of immune cells in the tumor microenvironment.

It is known that in addition to promoting T cell proliferation by antigen presentation, M1 type macrophages also play a key role in innate immunity and have a direct killing effect on tumors. We further examined the killing effect of macrophages in tumor tissue from KO mice. Consistent with the expectation, macrophages in the tumor microenvironment of KO mice were significantly more capable of killing B16 cells than those of WT mice (FIG. 18). Such result suggests that macrophages in the tumor microenvironment of melanoma were mostly type M1/killing capable macrophages upon FATS gene defect, such that the immune response for killing tumor cells was significantly stronger in KO mice than in WT mice, the increased polarization into M1 type macrophages directly affected the immune killing of tumor.

Macrophages polarization is a process of extreme complexity, in which NF-κB signaling pathway plays an important role according to studies. Duygu Sag et al. demonstrated that Abcgl gene defect can activate NF-κB signaling pathway and thereby promote the polarization into M1 type macrophages, and can also transform the macrophages in melanoma from tumor-promoting M2 type to tumor-suppressive M1 macrophages, such leading to significant inhibition on melanoma growth.

NF-κB is a nuclear transcription factor that regulates the expression of multiple genes. In the NF-κB signaling pathway, IκB family members (IκBα, Iκβ, etc.) bind to p65 (a key member of the NF-κB signaling pathway), keeping p65 in an inactive state. Various activation signals activate the NF-κB signaling pathway by degrading IκB. We performed WB assay on bone marrow-derived macrophages and found that p-p65 level was increased in type M0 macrophages of KO mice, that is, NF-κB signal was stronger in KO mice than in WT mice (FIG. 23), such suggesting that FATS gene defect can promote polarization into type M1 macrophage by activating NF-κB signaling pathway.

### Example 3 Experiment of FATS-KO Mouse Bone Marrow-Derived Macrophage Adoptive Therapy

### 3.1 Subjects and Methods

### 3.1.1 Experimental Methods

### 3.1.1.1 Establishment of a Subcutaneous Xenograft Model of Mouse Melanoma (Refer to 1.1.3.2)

### 3.1.1.2 Separation of Mouse Bone Marrow Cell (Referring to 2.1.3.4)

### 3.1.1.3 Directed Differentiation of Mouse Bone Marrow-Derived Macrophages (Refer to 2.1.3.5)

### 3.1.2 Method

10 female C57BL/6 mice, 6-8 weeks of age and weighing 18-20 g, were subcutaneously injected with B16 cells (2×10⁵ cells/mouse) to establish a xenograft model of subcutaneous melanoma (referring to 1.1.3.2 for specific procedures). The mice were randomly divided into two groups (5 mice per group). M0 type macrophages derived from the directed differentiation of bone marrow cells (stimulated with LPS for 12 hours) from the wild type and KO mice, were adoptively infused into the C57BL/6 mice on the 2nd and 7th day of tumor-graft, respectively. Tumor size was continuously monitored. The C57BL/6 mice were sacrificed 16 days after tumor-graft, and tumor tissue from the mice was weighed. Mononuclear cells were separated from the tumor tissue with mouse tumor tissue-infiltrating mononuclear cell separating solution. The separated mononuclear cells were analyzed by flow cytometry for the frequency of macrophages and M2 type macrophages in the tumor.

### 3.2 Results

### 3.2.1 Significant Inhibition of FATS Gene-Deficient Bone Marrow-Derived Macrophage Adoptive Therapy on Tumor Growth

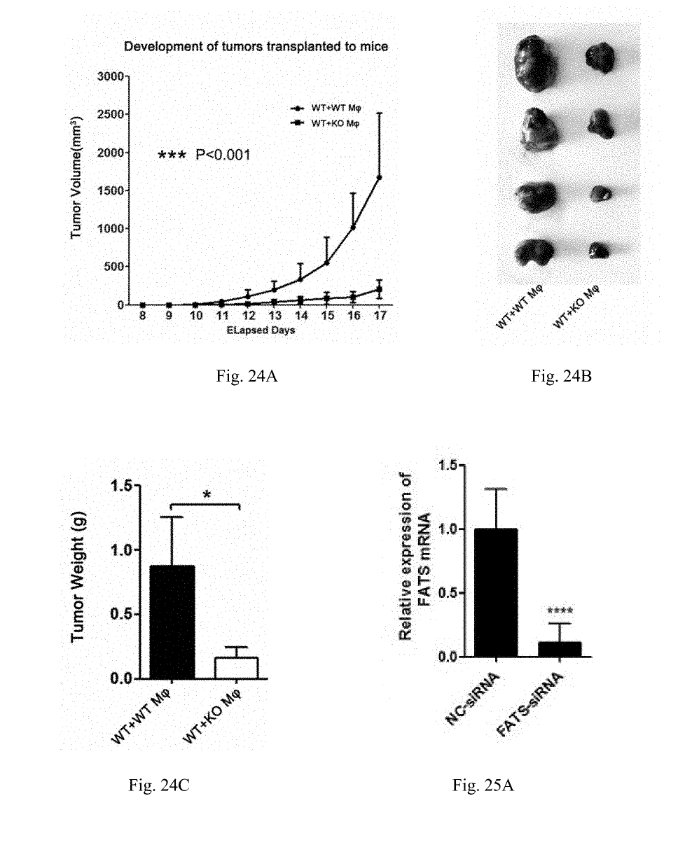

The result of the above experiment indicated that FATS gene deficient macrophages possess a crucial tumor-killing ability, thereby inhibiting tumor growth. To further confirm that FATS gene-deficient or FATs gene under-expressing macrophages inhibit melanoma growth, we injected wild type and FATS gene deficient or silenced macrophages into B16 cell tumor-bearing mice for an adoptive therapy of melanoma. The result showed that FATS gene deficient macrophages significantly inhibited melanoma growth after the adoptive therapy (FIG. 24A), with the final volume (FIG. 24B) and weight (FIG. 24) of the tumor significantly reduced.

### Example 4 Experiment of Adoptive Therapy Using Wild Type Mouse Bone Marrow-Derived Macrophages Transfected with FATS-siRNA

### 4.1 Subjects and Methods

### 4.1.1 Materials, Reagents and Instruments

### 4.1.1.1 Reagents

| | |
|---|---|
| Lipofectamine RNAiMAX Reagent | Invitrogen, US |
| (transfection reagent) | |
| FATS-siRNA | Guangzhou Ribo Biotech Co., Ltd. |

### 4.1.2 Experimental Methods

4.1.2.1 Directed Differentiation of Bone Marrow Cells into Macrophages (Referring to 2.1.3.4 and 2.1.3.5)
4.1.2.2. Macrophage Transfection with FATS-siRNA

Main materials and reagents: FATS-siRNA (synthesized by Guangzhou Ribo Biotech Co. Ltd, the corresponding DNA sequence is represented by SEQ ID NO: 2), Lipofectamine RNAiMAX Reagent (transfection reagent), PBS, RPMI1640 complete medium, and 12-well plates.

Method: the macrophages collected (in 4.1.2.1) were inoculated into a 12-well plate (5×10⁶ cells/well), with the cells concentrated at 60-80%. According to the table below, FATS-siRNA and Lipofectamine RNAiMAX Reagent were diluted with RPMI1640 complete medium and the diluted FATS-siRNA and Lipofectamine RNAiMAX Reagent was mixed in 1:1. The mixture was left standing for 5 minutes at room temperature and then the mixture was co-incubated with the cells for 1-3 days. The transfection efficiency was detected under microscope and a quantitative PCR was performed after 24 hours to further detect the transfection efficiency.

### Dilution of FATS-siRNA and Lipofectamine RNAiMAX Reagent

| Material/Reagent | 96-well plate | 24-well plate | 6-well plate |
|---|---|---|---|
| Adherent cells | (1-4) × 10⁴ | (0.5-2) × 10⁵ | (0.25-1) × 10⁶ |
| Medium for experiment | 25 μl | 50 μl | 150 μl |
| Lipofectamine RNAiMAX | 1.5 μl | 3 μl | 9 μl |
| Reagent | | | |
| Medium for experiment | 25 μl | 50 μl | 150 μl |
| siRNA | 1.5 μl | 3 μl | 9 μl |
| Diluted FATS-siRNA | 25 μl | 50 μl | 150 μl |
| Diluted Lipofectamine | 25 μl | 50 μl | 150 μl |
| RNAiMAX Reagent | | | |

### Preparation of siRNA-Lipid Mixture and Cell Transfection

| Material/Reagent | 96-well plate | | 24-well plate | | 6-well plate | |
|---|---|---|---|---|---|---|
| siRNA-lipid mixture | 10 | μl | 50 | μl | 250 | μl |
| Final concentration of | 1 | pmol | 5 | pmol | 25 | pmol |
| siRNA | | | | | | |
| Final concentration of | 0.3 | μl | 1.5 | μl | 7.5 | μl |

Lipofectamine
RNAiMAX Reagent

4.1.2.3 Induced Directed Polarization into Type M1 Macrophages

24 hours after the FATS-siRNA transfection, LPS (1 μg/ml) was added to induce macrophage polarization to M1 type macrophage. Cells were collected and counted after 12 hours, and the cell concentration was adjusted to 1×10⁷ cells/ml. The cells were analyzed by flow cytometry to determine the macrophage phenotype.

Note: macrophages transfected with empty vector were used as control.

### 4.1.2.4 M1 Type Macrophage Adoptive Therapy for Tumors

A mouse melanoma subcutaneous xenograft model was established (referring to 1.1.3.1 for specific procedures).

Specific methods: macrophages transfected with FATS-siRNA were injected into the tail vein of tumor-bearing mice on the 2nd and 7th day of tumor-graft (subcutaneous injection of B16 cells) for an adoptive therapy. The mice were sacrificed after 16 days and tumor size was monitored every other day during the tumor-graft.

Note: macrophages transfected with NC-siRNA were used as control.

### 4.2 Results

4.2.1 Adoptive Infusion of Bone Marrow-Derived Macrophages Transfected with FATS-siRNA Significantly Inhibits Tumor Growth

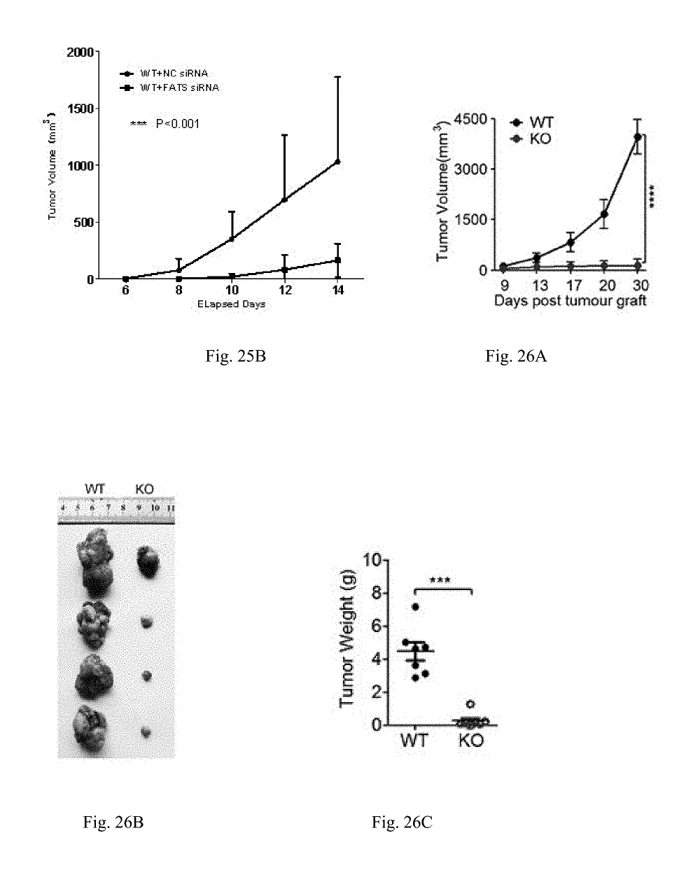

By using siRNA to silence the FATS gene in wild type macrophages, we transfected wild type macrophages with FATS/NC-siRNA, and subsequently treated the mice with melanoma with these two transfected macrophages. As shown in FIG. 25, FATS gene silenced macrophages significantly inhibited melanoma growth after the treatment. Such results confirm the therapeutic effect of FATS gene deficient or silenced macrophages on mouse melanoma.

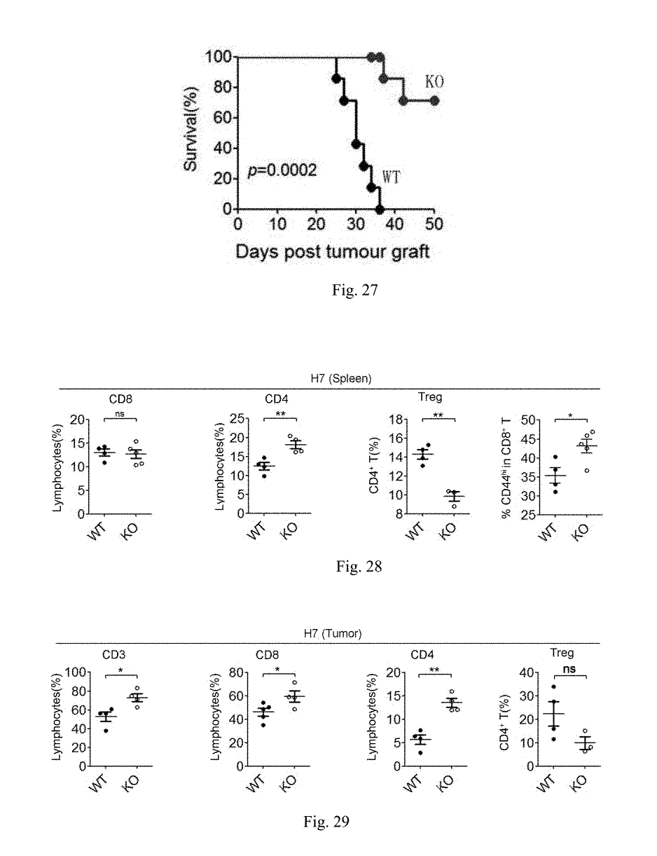

The above experiments and analysis and discussion thereof illustrated the correlation between FATS gene or its expression product and melanoma. In other experiments, we replaced melanoma with other tumors and further confirmed a same or similar correlation between FATS gene or its expression product and other tumors. Taking pancreatic cancer as an example, as shown in FIGS. 26-29, the growth of subcutaneous xenograft of pancreatic cancer in FATS gene-knockout mice was significantly inhibited, resulting in a significantly reduced weight of tumor, as well as prolonging the survival of the tumor-bearing mice. By detecting and analyzing the peripheral immune microenvironment of wild type and FATS gene-knockout mice, we found that the frequency of CD4⁺ T cells in the periphery increased, while the frequency of immunosuppressive Tregs decreased significantly; the frequency of CD8⁺ T cells in the periphery had no significant increase, while the proportion of CD44^{hi} in CD8⁺ T cells increased significantly, indicating a significantly enhanced activity of CD8⁺ T cells. Meanwhile, we also detected and analyzed the changes of immune cells in the tumor immune microenvironment that play a key role in tumorigenesis and progression of tumor; wherein we found that the frequency of total T cells and CD4⁺ T cells in tumor tissue increased significantly, while the frequency of Treg cells did not, such indicating an increase in Th1 cells frequency. We also noticed an increase in CTL frequency in tumor tissue. Generally, through the above analysis we believe that FATS gene knockout enhanced the activity of CD8⁺ T cells by reducing the frequency of immunosuppressive cells in peripheral tissues, while inhibiting tumor growth by increasing the frequency of CTLs in tumor tissue. The experimental results have suggested the availability of FATS gene as a potential target for immunotherapy that can largely improved the therapeutic effect of immunotherapy by regulating the frequency and function of T cells in peripheral and tumor tissues.

In other words, the knockout or inhibition of FATS gene can regulate the macrophage polarization to M1 type and the increase and activation of T cells, thereby inhibiting tumor growth. This can be used to adjust M1 type macrophage polarization and T cells increase and activation.

It should be noted, that the above description is used only to illustrate the preferred embodiments of the present invention with no intention of limiting the scope thereof. Any modification, replacement, improvement, etc., made without departing from the principle of the invention, should also be considered within the scope of the present invention.

What is claimed is:

1. A use of FATS gene, by knocking out or inhibiting the FATS gene, in promoting macrophage polarization into M1 type and/or inhibiting macrophage polarization into M2 type, or activating and proliferating killer T cells.

2. A use of FATS gene or an expression product thereof in any one of:
i) developing and screening a functional product for tumors, and
ii) preparing a functional product for treatment or prevention of tumors.

3. The use of claim 2, wherein the functional product is a product or a latent substance at least for treating, alleviating, inhibiting or regulating occurrence and progression of tumors.

4. The use of claim 2, wherein the functional product at least down-regulating expression, transcription of the FATS gene or expression product of the FATS gene.

5. The use of claim 2, wherein the functional product increases infiltration of inflammatory cell in a tumor tissue.

6. The use of claim 2, wherein the functional product promotes an anti-tumor immunity and/or inhibits a tumor-promoting immune response in a peripheral immune organ or a tumor immune microenvironment.

7. The use of claim 2, wherein the functional product promotes polarization of macrophages into M1 type and/or inhibits polarization of macrophages into M2 type during differentiation of macrophages.

8. The use of claim 2, wherein the functional product is used for one or more of:
i) increasing frequency of at least one of cytotoxic T lymphocytes, NK cells, γδT cells, and M1 type macrophages;
ii) reducing a frequency of regulatory T lymphocytes and/or M2 type macrophages;
iii) increasing an expression of cytokine IL-12 secreted by M1 type macrophages and/or an expression of T cell proliferation-associated cytokine IL-2;
iv) increasing a killing ability of macrophages;
v) increasing a proliferative ability of T cells;
vi) reducing an expression of VEGF by macrophages;
vii) inhibiting angiogenesis in a tumor tissue;
viii) promoting polarization of macrophages into M1 type and/or inhibiting polarization of macrophages into M2 type during differentiation of bone marrow cells into macrophages.
ix) promoting an apoptosis of M2 type macrophages; and
x) activating a NF-κB signaling pathway.

9. The use of claim 2, wherein the functional product is selected from or comprises one or more of:
a nucleic acid inhibitor, a protein inhibitor, an antibody, a ligand, a proteolytic enzyme, a protein-binding molecule, a FATS gene deficient or silenced immune-associated cell or a differentiated cell or a construct thereof, capable of down-regulating expression or expression product of the FATS gene at a genetic or protein level.

10. The use of claim 2, wherein the functional product is selected from or comprises one or more of:
a small interfering RNA, a dsRNA, a shRNA, a microRNA or an antisense nucleic acid targeting the FATS gene or a transcript of the FATS gene and capable of inhibiting expression or transcription of the FATS gene; and a construct capable of expressing or forming the small interfering RNA, the dsRNA, the shRNA, the microRNA or the antisense nucleic acid.

11. The use of claim 2, wherein the functional product is selected from comprises any one of:
i) a small interfering RNA, a dsRNA, an shRNA, a microRNA or an antisense nucleic acid targeting SEQ ID NO:1 or a transcript of SEQ ID NO:1 and capable of inhibiting expression or transcription of the FATS gene;
ii) a construct capable of expressing or forming the small interfering RNA, the dsRNA, the shRNA, the microRNA or the antisense nucleic acid in i);
iii) a construct, comprising SEQ ID NO: 1 or a complementary sequence of SEQ ID NO: 1, and capable of forming an interfering molecule inhibiting expression or transcription of the FATS gene in vivo;
iv) an immune-associated cell in which SEQ ID NO: 1 is inhibited or knocked out, or a differentiated cell or a construct of the immune-associated cell;
v) a small interfering RNA, a dsRNA, an shRNA, a microRNA or an antisense nucleic acid targeting a homologous sequence or a transcript of SEQ ID NO: 1 according to codon preference in organism of a construct, and capable of inhibiting expression or transcription of the FATS gene;
vi) a construct capable of expressing or forming the small interfering RNA, the dsRNA, the shRNA, the microRNA or the antisense nucleic acid in v);
vii) a construct comprising a homologous sequence or a complementary sequence of SEQ ID NO: 1 according to codon preference of organism of the construct, and capable of forming an interfering molecule inhibiting expression or transcription of the FATS gene in vivo; and
viii) an immune-associated cell in which a homologous gene sequence of SEQ ID NO: 1 according to codon preference of organism of a construct is inhibited or knocked out, or a differentiated cell or a construct of the immune-associated cell.

12. The use of claim 2, wherein the expression product of the FATS gene comprises a protein encoded by the FATS gene.

13. The use of claim 2, wherein the tumors comprise melanoma or pancreatic cancer.
